(19) United States
(12) Patent Application Publication
(10) Pub. No.: US 2024/0186001 A1
(43) Pub. Date: Jun. 6, 2024

(54) SYSTEMS AND METHODS FOR DETECTING A DISEASE CONDITION

(71) Applicant: Icahn School of Medicine at Mount Sinai, New York, NY (US)

(72) Inventors: John Martignetti, Chappaqua, NY (US); Peter Dottino, New York, NY (US); Boris Reva, New York, NY (US)

(21) Appl. No.: 17/769,486

(22) PCT Filed: Oct. 16, 2020
(86) PCT No.: PCT/US20/56170

§ 371 Date: Apr. 15, 2022

Related U.S. Application Data
(60) Provisional application No. 62/916,103, filed on Oct. 16, 2019.

(51) Int. Cl. G16H 50/20; G01N 33/574; G16B 20/00; G16H 50/30

(52) U.S. Cl. CPC G16H 50/20; G01N 33/57442; G01N 33/57449; G16B 20/00; G16H 50/30

(57) ABSTRACT

Systems and methods for evaluating a gynecological disorder in a subject is disclosed. A biological fluid sample is obtained from the subject. Protein fractions are purified from the biological fluid sample, thereby obtaining a protein preparation. For each protein in a set of proteins, a corresponding abundance value for the respective protein in the protein preparation is determined, thereby obtaining a protein abundance dataset for the subject. Using the protein abundance dataset, values for each of a set of protein abundance features are determined, thereby obtaining a feature dataset for the subject. The feature set is input into a classifier. The classifier is trained to distinguish between at least two states of the gynecological disorder based on at least the set of protein abundance features, thereby obtaining a probability or likelihood from the classifier that the subject has a particular state of a gynecological disorder.

### CROSS REFERENCE TO RELATED APPLICATION

This application claims priority to U.S. Provisional Patent Application No. 62/916,103, entitled “Systems and Methods for Detecting a Disease Condition,” filed Oct. 16, 2019, which is hereby incorporated by reference.

### TECHNICAL FIELD

This specification describes a system using proteomic analysis to evaluate subjects for having a disease condition. It is based upon the collection of a biological sample, proteomic characterization of the sample, and application of a machine learning approach to assign a risk score between two different states of disease.

### BACKGROUND

Cancer is a leading cause of death worldwide. Given that early stage solid cancers, those that are still localized to their site of origin, can generally be cured by surgery alone (see Siegel et al., 2018 C A Cancer J Clin 68, 7-30), a major focus of cancer research has been detection of premetastatic and early stage cancer lesions.

One-third of all women of reproductive age will experience nonmenstrual pelvic pain at some point in their lives (see Stratton 2020 UpToDate 5473 and Am College Obst. Gyn. 2020 Obstet Gynecol 135, e98-e109) and one-third of outpatient visits to gynecologists in the U.S. are for evaluation of abnormal uterine bleeding (see Kaunitz 2020 UpToDate 3263). For many women, these symptoms accompany infertility which is reported in ˜10% of all US women and even higher percentages worldwide. See e.g. Wilkes et al. 2009 Family Practice 26, 269-274; Am College Obst. Gyn. 2019 Obstet Gynecol 133, e377-e384; and Stahlman 2019 Msmr 26, 20-27. For almost all of these women, these conditions result in a diagnostic odyssey wherein women struggle through multiple physicians over many years for a definitive diagnosis. See Nnoaham et al. 2011 Fertil Steril 96, 366-373; Ballard et al. 2006 Fertil Steril 86, 1296-1301; and Zondervan et al. 2020 N Engl J Med 382, 1244-1256.

In general, the diagnostic algorithm for pelvic pain, abnormal bleeding, and infertility begins with a detailed history and physical exam, followed by laboratory tests and imaging. Frequently the results from these tests are inconclusive, and women will need to undergo laparoscopy or hysteroscopy with dilation and curettage (D&C) for definitive diagnosis. Indeed, more than 198,000 operating room (OR)-based hysteroscopies are performed each year in the U.S. (see Hall et al 2017 Natl Health Stat Report 1-15 and Tam et al. 2016 J Min Invasive Gyn 23, S194), costing an average $14,600 per procedure or $2.9 B/year. OR-based hysteroscopy is performed under anesthesia by a surgeon and is associated with pain, risks of general anesthesia, and, indirectly, loss of time at work for the patient.

Ovarian and endometrial cancers are cancers for which early detection would be expected to significantly increase survival. Typically, these cancers are first diagnosed at a late stage and exhibit aggressive phenotypes with poor survival rates. See Ledermann et al. et al. 2013 Annals of Oncology 24(Supplement 6), vi24-vi32 and Colombo et al. et al. 2011 Annals of Oncology 22(Supplement 6), vi35-vi39. For example, of all cases of ovarian cancer diagnosed each year, approximately 75% are classified at diagnosis as high-grade serous cancers, which have a poor prognosis, with a 5-year survival rate of 10% to 30%. See e.g., Bodurka et al 2012 Cancer, 3087-3094.

At present, there are no screening tests for ovarian or endometrial pre-metastatic lesions or cancer. Typically, patients are tested only after they present with symptoms, when the cancer is advanced and prognosis is poor, and existing test methods suffer in both sensitivity and specificity. See Nair et al., 2016 PLoS Med 13(12):e1002206.

There will be more than 80,000 diagnoses of ovarian (OvCA) and endometrial (EndoCA) cancers this year in the U.S., and it is estimated that they will result in the death of 26,000 women. Cancer stage at diagnosis directly dictates treatment options and is the primary determinant of overall survival. For both of these gynecologic cancers, detection of early-stage, localized disease is associated with 5-year survival rates over 90%, while diagnosis with late-stage, metastatic disease results in dramatically reduced 5-year survival rates of ˜25%. Nearly 80% of OvCA cases are detected in late stages when the cancer has already spread. Twenty-five % of women diagnosed with EndoCA have late-stage disease. OvCA, in particular, often progresses without overt symptoms and presents later in the course of disease with non-specific symptoms (for example, constipation or diarrhea). Diagnosis requires radiographic imaging (transvaginal and/or abdominal ultrasonography, CT, MRI and/or PET) followed by radical cytoreductive surgery. In addition, these cancers disproportionally affect ethnically distinct populations. For example, 5-year survival rates for white and black women with EndoCA are 84% and 62%, respectively. Black women are also less likely to be correctly diagnosed with early-stage disease, and their survival rate at every stage is lower. Similar poorer outcomes are present in black women with OvCA. For all women, there are no screening tests for either of these two cancers or their known precursors, making detection at their earliest and curable stages nearly impossible.

### SUMMARY

Accordingly, there is a need for screening tests for solid tumors that provide greater sensitivity and specificity, that can detect precancerous changes, and that would allow diagnosis of solid tumors when still at a stage suitable for cure by surgical resection. There is a particular need for screening tests for endometrial and ovarian cancer. The present disclosure addresses the shortcomings identified in the background by providing robust techniques for detecting whether a subject has a disease condition, e.g., cancer.

There are no diagnostic or screening tools to detect OvCA in its early, curable stages. Without this critical ability for earlier detection, 80% of OvCA cases will continue to be detected after the cancer has spread and 5-year survival is <25%. Similarly, when OvCA is detected in later stages there are no prognostic tools to predict which women will respond to the current platinum-based, first-line treatment. An protein-based diagnostic test could help immediately triage women to receive the most appropriate treatments without needless co-morbidities secondary to wasted time and chemotherapy side-effects. Given the lethality and quality-of-life differences between early- and late-stage OvCA and the different treatment, management and maintenance options becoming available, the methods described herein use an OvCA molecular panel to provide actionable information to guide patient management.

In some embodiments, a single diagnostic test is provided for simultaneous screening for OvCA and EndoCA in asymptomatic women. In some embodiments, the test will consist of detection of a panel of proteins enriched from a biological fluid sample, e.g., a uterine lavage sample, that together can distinguish between: (1) women with and without cancer, (2) OvCA (requiring surgery) from EndoCA (potential for no or minimal surgical management), and (3) less and more aggressive EndoCA (none vs more extensive surgical treatment and chemotherapy).

In some embodiments, the diagnostic assay described herein is based on a new proprietary application of a ML-based method for classification of molecular profiles. The underlying mathematic model allows the combination of imperfect signals of individual biomarkers into a significantly more powerful classification function that can differentiate molecular profiles of biologically different tumors or biospecimens. While the parent approach used gene expression levels as biomarkers, the current application will implement a new proprietary approach. In some embodiments, it replaces gene biomarkers with entropy-based scoring of the position of subsets of differentially expressed proteins in a sample-specific ranked list of proteins. This approach helps avoid batch effects because it uses relative expression values, rather than absolute values and significantly reduces the number of biomarkers that will be required for the commercial diagnostic panel. Classification accuracies have been compared with accuracies produced by 10 other well-established machine learning algorithms including Support Vector Machine and Random Forest. The current ML approach produced the most accurate classifications.

In accordance with some embodiments, a method for evaluating a gynecological disorder in a subject includes obtaining a first biological fluid sample from the subject. The method includes enriching a protein fraction from the first biological fluid sample, thereby obtaining a first protein preparation. The method includes determining, for each protein in a first set of proteins, a corresponding abundance value for the respective protein in the protein preparation. The method thereby includes obtaining a first protein abundance dataset for the subject. The method includes determining, using the first protein abundance dataset, values for each of a first set of protein abundance features. The method thereby includes obtaining a first feature dataset for the subject. The method also includes inputting the first feature set into a classifier. The classifier is trained to distinguish between at least two states of the gynecological disorder based on at least the first set of protein abundance features. The method thereby includes obtaining a probability or likelihood from the classifier that the subject has a particular state of a gynecological disorder.

Another aspect includes a non-transitory computer readable storage medium and one or more computer programs embedded therein, the one or more computer programs comprising instructions which, when executed by a computer system, cause the computer system to perform the method. An additional aspect includes a device comprising one or more processors, and memory storing one or more programs for execution by the one or more processors.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is a block diagram illustrating an example of a computing system in accordance with some embodiments of the present disclosure.

FIGS. 2A, 2B, and 2C are prior art from Rykunov et al 2016 Nuc Acids Res 44(11), e110 illustrating a) the selection of nominated driver genes associated with cancer type, b) ranking of autoantibodies in terms of significance and occurrence, and c) determining a molecular signature of a disease based on classification accuracy.

FIGS. 3A and 3B collectively illustrate the classification of patient samples derived from blood plasma with regard to polyp diagnoses, in accordance with some embodiments of the present disclosure.

FIGS. 4A and 4B collectively illustrate the classification of patient samples derived from uterine lavage with regard to polyp diagnoses, in accordance with some embodiments of the present disclosure.

FIGS. 5A, 5B, and 5C collectively illustrate the classification of patient samples derived from blood plasma with regard to endometrial cancer, in accordance with some embodiments of the present disclosure. The classification accuracies were assessed by areas under receiver operating curve (AUC-ROC) (e.g., FIG. 5A). The presented characteristics were derived from 4000 individual classification tests, where the original data set of 30 EndoCA and 30 benign control samples was divided by random in training and test sets each of ˜50% of samples (˜15 cancer and ˜15 benign samples). The training set was used to determine biomarkers (differentially abundant proteins) which were used to compute a classification scoring function (weighted sum of biomarkers' expression values) that was constructed to optimize separation of the training set into given clinical classes. Samples in the test set were then classified using the classification function of the training set (i.e. biomarkers, biomarker weights and classification threshold). Thus, in each classification test, each sample was classified in one of the given classes (training or test sets) and each sample was assessed by classification score. FIGS. 5B and 5C illustrate averaged classification probabilities as functions of averaged scoring functions. The classification accuracy depends on scoring function and increases at the tails of the distribution.

FIGS. 6A, 6B, and 6C collectively illustrate the classification of patient samples derived from uterine lavage with regards to endometrial cancer, in accordance with some embodiments of the present disclosure. FIGS. 6A-6C are derived from the same initial data as FIG. 5A-5C.

FIG. 7 illustrates an overview of the method of evaluating a gynecological disorder in a subject in accordance with some embodiments of the present disclosure.

FIGS. 8B and 8C collectively illustrate the classification of patient samples derived from uterine lavage with regard to polyp diagnoses, in accordance with some embodiments of the present disclosure.

FIGS. 9A and 9B collectively illustrate the classification of patient samples derived from blood plasma with regard to polyp diagnoses, in accordance with some embodiments of the present disclosure.

### DETAILED DESCRIPTION

There is a clear unmet need for a simple screening test to detect epithelial ovarian cancer (OvCA) prior to symptom onset and its ultimate spread. OvCA develops and progresses without overt symptoms and presents even at late stages with non-specific symptoms. Detection of early-stage, localized disease is associated with 5-year survival rates which exceed 90%. Diagnosis at late-stage, metastatic disease results in dramatically reduced 5-year survival rates of less than 25%. Currently, nearly 80% of OvCA cases are detected in late stages when the cancer has already spread. Current methods of OvCA diagnosis are inadequate for detecting early stage disease and there are no screening tools for this cancer. In addition, while 80% of women treated for later stage disease are determined by current technologies to have had a complete clinical response to their primary therapy, the majority will die from disease recurrence/chemoresistance within 5 years and it is impossible to distinguish who will respond and who will not. Thus, throughout the arc of a patient's clinical care, there is a clear but unmet need for new diagnostic technologies that can (1) detect OvCA in its earliest stages and (2) provide prognostic information regarding treatment and/or outcome response for those diagnosed at later stages.

Based on the current lack of biomarkers, no screening programs exist or are currently recommended for these two cancers. Two large, randomized controlled trials (PLCO, n=78,00071,72 and UKCTOCS, n=202,63873) have investigated the potential of using a combination of cancer antigen 125 (CA 125) and transvaginal ultrasound (TVU) for OvCA screening; however, OvCA mortality was not significantly different between intervention and control groups. Based on the failures of these two trials, and a lack of alternate, effective novel biomarkers/diagnostics, the US Preventative Services Task Force recommends against OvCA screening.

Given the limitations of the currently available approaches, efforts continue to search for new screening biomarkers. The most effective tests under development incorporate multiple biomarkers. A subset of samples from the UKCTOCS study (n=80 women) were analyzed and 5 additional longitudinal biomarkers were identified that together improve upon CA. A test called PapSEEK that analyzes DNA in fluids obtained during a Pap test detects mutations in 18 genes and assesses aneuploidy; however, PapSEEK only displayed a sensitivity of 33% for early-stage ovarian cancer (specificity of ˜99%) when used alone (n=245 women with OvCA; 382 with EndoCA). The sensitivity increased to 63% (95% CI, 51 to 73%) when combined with plasma biochemical testing. While a number of approaches demonstrate relatively good detection of late-stage cancers these tests remain unsatisfactory for early-stage/pre-metastatic detection. As noted above, detection of early-stage cancers offers the opportunity for improved treatments and outcomes. There are a number of registered clinical trials currently recruiting or active; however, many are in the discovery phase and involve approaches not ideal for development of screening tests for early-stage identification such as mass spectrometry, or collection of samples under anesthesia. Tests that rely exclusively on identification of cancer mutations are also unlikely to be effective for screening. Published and unpublished studies from our group and others using next-generation sequencing of cellular and cell-free DNA collected from uterine lavage, tissue samples, and blood revealed a previously unknown and prevalent landscape of cancer driver mutations in women without cancer, illuminating the need for additional information beyond DNA mutation analysis.

Such diagnostic technologies would dramatically change clinical management and treatment and save tens of thousands of lives worldwide each year. To address this need, we have been leveraging access to >12 years of longitudinally collected and deeply annotated biobanked plasma and uterine lavage samples from the Gynecologic Cancer Translational Research Program (GCTRP; Icahn School of Medicine at Mount Sinai; New York, NY and Nuvance Health, Danbury, CT) to develop a liquid-biopsy based diagnostic test. Originally, using a genomics-based approach, we and others demonstrated the ability to detect OvCA using circulating tumor DNA (ctDNA); however, we demonstrated a previously unknown and prevalent landscape of cancer driver mutations in women without cancer. Our findings have since been independently confirmed and highlighted, illuminating the need for complementary information beyond DNA mutation analysis.

To overcome these challenges, multiple-biomarker screening assays have been developed that use proteomic information, e.g., using exosomal preparations from biological fluids. This approach is unique in that we have access to a rich source of matched blood and uterine lavage samples with accompanying longitudinal clinical information and, importantly, clinically-relevant control populations. We have pioneered the use of uterine lavage as a powerful, and anatomically-relevant analyte for earliest detection of gynecologic malignancies and, as detailed in this application, further demonstrate its unique advantages for proteomic profiling. We are using powerful/innovative methods for biomarker discovery. (1) protein fraction enrichment and mass-spectrometry (MS) analysis which overcomes multiple limitations in current studies. (2) The combination of both plasma and uterine lavage fluid. Lavage fluid offers direct contact with the anatomic source of OvCA and represents a powerful biofluid for gynecologic cancer biomarker discovery. (3) A novel machine learning (ML) algorithm to construct classification scoring functions for detection and clinical classification of OvCA with high confidence. This will facilitate development of a commercial diagnostic test to challenge current clinical practice by enabling screening for OvCA in asymptomatic women and provide prognostic information regarding treatment and outcome for those harboring late stage disease. Accordingly, as described herein, OvCA proteomic signatures derived from protein preparations, of both tumor and microenvironment origin, can be used to derive sensitive and specific diagnostic and prognostic OvCA biomarkers.

Gynecologic diseases are those diseases that involve the female reproductive track. These diseases and health conditions include both benign and malignant tumors including endometrial and ovarian cancers; premalignant conditions such as endometrial hyperplasia and cervical dysplasia, benign (i.e. non-cancerous conditions) including polyps, ovarian cysts, fibroids and adenomyosis; endometriosis (the implantation of ectopic endometrial tissue outside the uterus, resulting in symptoms including infertility, dysmenorrhea and pelvic pain), pregnancy-related diseases and infertility, menopause, pelvic inflammatory diseases and infection, and even endocrine diseases which relate to the female reproductive tract, for example primary and secondary amenorrhea, polycystic ovary syndrome and premature ovarian failure.

The distinct gynecologic diseases may themselves have broader downstream health ramifications which result in diagnostic odysseys taking up years of physicians visits and a range of diagnostic tests. For example, one-third of all women of reproductive age will experience nonmenstrual pelvic pain at some point in their lives [Stratton, P. (2020). Evaluation of acute pelvic pain in nonpregnant adult women. UpToDate 5473. PMID.; American College of Obstetricians and Gynecologists. (2020). Chronic Pelvic Pain: ACOG Practice Bulletin, Number 218. Obstet Gynecol 135, e98-e109. PMID: 32080051.] and one-third of outpatient visits to gynecologists in the United States are for evaluation of abnormal uterine bleeding [Kauntiz, A. M. (2020). Approach to abnormal uterine bleeding in nonpregnant reproductive-age women. UpToDate 3263.] These two non-specific symptoms, pelvic pain and abnormal bleeding, can be caused by a wide variety of non-pregnancy related conditions, including endometrial polyps, leiomyomas (uterine fibroids), adenomyosis, endometriosis, gynecological cancer, or pelvic inflammatory disease, among others. For many women, a number of these conditions also result in infertility which is reported in ˜10% of all US women and even higher percentages worldwide [Wilkes, S., Chinn, D. J., Murdoch, A. & Rubin, G. (2009). Epidemiology and management of infertility: a population-based study in UK primary care. Family practice 26, 269-274; Centers for Disease Control and Prevention. National Center for Health Statistics: Infertility, https://www.cdc.gov/nchs/fastats/infertility.htm; American College of Obstetricians and Gynecologists. (2019). Infertility Workup for the Women's Health Specialist: ACOG Committee Opinion, Number 781. Obstet Gynecol 133, e377-e384. PMID: 31135764.; Stahlman, S. & Fan, M. (2019). Female infertility, active component service women, U.S. Armed Forces, 2013-2018. Msmr 26, 20-27. PMID: 31237765.]

For almost all of these women, these conditions result in a diagnostic odyssey wherein women struggle through multiple physicians over many years for a definitive diagnosis. For example, on average, women with endometriosis consult seven physicians prior to diagnosis [Nnoaham, K. E., Hummelshoj, L., Webster, P. et al. (2011). Impact of endometriosis on quality of life and work productivity: a multicenter study across ten countries. Fertil Steril 96, 366-373.e368. EMS48415. PMC3679489; Ballard, K., Lowton, K. & Wright, J. (2006). What's the delay? A qualitative study of women's experiences of reaching a diagnosis of endometriosis. Fertil Steril 86, 1296-1301. PMID: 17070183; Zondervan, K. T., Becker, C. M. & Missmer, S. A. (2020). Endometriosis. N Engl J Med 382, 1244-1256. PMID: 32212520].

In general, the diagnostic algorithm for pelvic pain, abnormal bleeding and infertility begins with a detailed history and physical exam, followed by laboratory tests and imaging (sonohysterogram, transvaginal and transabdominal ultrasound, MRI). Frequently the results from these tests are inconclusive, and women will need to undergo laparoscopy or hysteroscopy with dilation and curettage (D&C) for definitive diagnosis. Indeed, >198,000 operating room (OR)-based hysteroscopies are performed each year in the U.S. [Hall, M. J., Schwartzman, A., Zhang, J. & Liu, X. (2017). Ambulatory Surgery Data From Hospitals and Ambulatory Surgery Centers: United States, 2010. Natl Health Stat Report, 1-15. PMID: 28256998; Tam, T., Archill, V. & Lizon, C. (2016). Cost Analysis of In-Office versus Hospital Hysteroscopy. Journal of minimally invasive gynecology 23, S194], costing an average $14,600 per procedure or $2.9 B/year. OR-based hysteroscopy is performed under anesthesia by a surgeon and is associated with pain, risks of general anesthesia, and indirectly, loss of time at work for the patient. Having a diagnostic test

A number of these common gynecologic conditions also disproportionally affect ethnically distinct populations. For example, leiomyomas are 3× more prevalent in Black women and these leiomyomas may be larger and more numerous causing worse symptoms and greater surgical complications [Baird, D. D., Dunson, D. B., Hill, M. C., Cousins, D. & Schectman, J. M. (2003). High cumulative incidence of uterine leiomyoma in black and white women: ultrasound evidence. Am J Obstet Gynecol 188, 100-107. PMID: 12548202; Marshall, L. M., Spiegelman, D., Barbieri, R. L. et al. (1997). Variation in the incidence of uterine leiomyoma among premenopausal women by age and race. Obstetrics & Gynecology 90, 967-973.; Faerstein, E., Szklo, M. & Rosenshein, N. (2001). Risk factors for uterine leiomyoma: a practice-based case-control study. I. African-American heritage, reproductive history, body size, and smoking. Am J Epidemiol 153, 1-10. PMID: 11159139].

In some embodiments, the methods described herein provides a diagnostic risk score, based on either blood and/or uterine lavage fluid analysis, that can identify an underlying gynecologic disease. This disease can be present in either an asymptomatic (i.e. a screening test) or a symptomatic (i.e. a diagnostic test) woman. These diagnostic risk scores will provide clinically actionable information in the form of guidance towards disease-specific treatment.

For example, for a female who is experiencing acute or chronic pelvic or abdominal pain, uterine bleeding, and/or infertility part of their current gold-standard diagnostic evaluation today by either their internist, general practitioner, reproductive specialist or gynecologist could require radiologic (CT, MRI, PET scan, transabdominal ultrasound) examination coupled with invasive operating room-based tissue biopsy (dilation and curettage; D&C) for diagnosis. In this context, and instead using our method at the start of a patient's diagnostic evaluation, a blood sample and/or uterine lavage fluid sample would be obtained for analysis. Depending on the disease identified, clinically actionable information in the form of guidance towards disease-specific treatment would then be delivered by the method's risk score. For example, if a risk score suggesting endometriosis was identified by the blood and/or uterine lavage-based test, the patient could avoid the need for additional diagnostic procedures including ultrasound evaluation, MM and surgical laparoscopy. Instead, with our liquid biopsy based diagnosis, medical management for pain could be provided as well as medical management to directly treat the underlying disease, endometriosis. Medical management, avoiding surgery, could include the use of hormonal contraceptives, gonadotropin-releasing hormone (Gn-RH) agonists and antagonists, progestin therapy and aromatase inhibitors. Thus, in this example of a symptomatic patient of unknown disease etiology, the use of our method provides clinically actionable information capable of guiding day-to-day decision-making. It avoids the necessity for radiologic and surgical interventions to generate a diagnosis. Moreover, our method provides an opportunity to treat a gynecologic disease with medical management instead of surgical intervention which has historically included surgery to remove the uterus (hysterectomy) and both ovaries (oophorectomy).

Alternatively, if the diagnostic method identified a high risk score for ovarian cancer, that patient would be immediately sent from their internist, general practitioner, reproductive specialist or gynecologist to a specialist in diagnosing and treating gynecologic cancers. The directed transfer of care from a generalist practitioner to a cancer specialist would save time, avoid the intervening use of non-critical and expensive examinations, and as has been shown, treatment of women with gynecologic cancers by gynecologic oncologists and in specialized centers results in markedly improved outcomes for the patient [doi: 10.1016/j.ygyno.2007.02.030; doi: 10.1093/jnci/djj019; doi: 10.1097/01.AOG.0000265207.27755 0.28]

Finally, and given the costs of the diagnostic tests involved, inequalities of healthcare distribution, the limited geographic availability of and disproportionate distribution of the expertise/cost of trained operators/skilled physicians and equipment for diagnostic testing, our biomarker method requiring a blood sample or uterine lavage has the capacity to be performed in a general practitioners' office, performed by physicians' assistants or nurse practitioners, thus democratizing the overall diagnostic experience.

Development of a minimally invasive test that will efficiently diagnose the cause of these non-specific symptoms or triages women most likely to benefit from hysteroscopy or other invasive definitive testing would simultaneously minimize diagnostic delays, unnecessary surgeries, and possible loss of fertility, while improving outcomes and multiple burdens on the healthcare system. The methods described herein provide for a diagnostic test used to detect disease conditions in subjects. Particularly relevant disease conditions are early stage endometrial and ovarian cancers. Specifically, the methods enable testing a biological sample (e.g., lavage fluid) from a patient to distinguish between two or more different disease conditions, in particular between ovarian and endometrial cancer or between ovarian and/or ovarian cancer and non-cancer (e.g., evaluate a subject for a stage of a particular cancer condition or evaluate a subject for cancer vs non-cancer). In some embodiments, the methods described herein also provide for testing a biological sample to determine a probability or likelihood that a patient has a disease condition. In some embodiments, the method determines a probability or likelihood that a patient has a cancer of the uterus and/or female reproductive system (e.g., endometrial, cervical, or ovarian cancer). In some embodiments, the method determines a probability or likelihood that a patient has a non-cancerous disease of the uterus and/or female reproductive system (e.g., endometriosis, polyps, etc.).

The methods described herein provide for a diagnostic test used to detect disease conditions in subjects. Particularly relevant disease conditions are early stage endometrial and ovarian cancers. Specifically, the methods enable testing a biological sample (e.g., lavage fluid) from a patient to distinguish between two or more different disease conditions, in particular between ovarian and endometrial cancer or between ovarian and/or ovarian cancer and non-cancer (e.g., evaluate a subject for a stage of a particular cancer condition or evaluate a subject for cancer vs non-cancer). In some embodiments, the methods described herein also provide for testing a biological sample to determine a probability or likelihood that a patient has a disease condition. In some embodiments, the method determines a probability or likelihood that a patient has a cancer of the uterus and/or female reproductive system (e.g., endometrial, cervical, or ovarian cancer). In some embodiments, the method determines a probability or likelihood that a patient has a non-cancerous disease of the uterus and/or female reproductive system (e.g., endometriosis, polyps, etc.).

This invention analyzes biological samples, such as lavage analytes, by combining screening for protein biomarkers, for example using mass spectroscopy, with a novel computational classifier. The methods described herein can be used for evaluation of disease conditions in both symptomatic and asymptomatic individuals (e.g., a patient does not need to exhibit one or more symptoms of ovarian or endometrial cancers). In particular, these methods can be performed as part of an annual or other screening (e.g., concurrent with a pap or STD test). Through early detection of many disease conditions, patients can receive appropriate treatment sooner. For some cancers in particular, for example ovarian and endometrial cancers, early detection contributes to significant increases in survival rates of patients.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

Unless defined otherwise, all technical and scientific terms used herein have the meaning commonly understood by a person skilled in the art to which this invention belongs. The following references provide one of ordinary skill in the art with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991); Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the presently disclosed subject matter. As used herein, the following terms have the meanings ascribed to them below, unless specified otherwise.

As used herein, “gynecologic diseases” are those diseases that involve the female reproductive track. These diseases and health conditions include both benign and malignant tumors including endometrial and ovarian cancers; premalignant conditions such as endometrial hyperplasia and cervical dysplasia, benign (i.e. non-cancerous conditions) including polyps, ovarian cysts, fibroids and adenomyosis; endometriosis (the implantation of ectopic endometrial tissue outside the uterus, resulting in symptoms including infertility, dysmenorrhea and pelvic pain), pregnancy-related diseases and infertility, menopause, pelvic inflammatory diseases and infection, and even endocrine diseases which relate to the female reproductive tract, for example primary and secondary amenorrhea, polycystic ovary syndrome and premature ovarian failure.

As used herein, the term “lavage fluid” refers to a biological sample that is collected from a body cavity of a subject. In particular, “uterine lavage fluid” refers to a biological sample collected from a subject's uterus (e.g., via one or more washings). Lavage fluid can be used to test or screen for one or more disease conditions. See e.g., Nair et al., 2016 PLoS Med 13(12):e1002206 and Meyer et al. et al. 2011 Eur Respir J 38, 761-769. In certain circumstances, the use of lavage fluid is a less invasive method of screening for disease (e.g., as compared to other biopsy methods).

As used herein, the term “mutation” refers to permanent change in the DNA sequence that makes up a gene. In certain embodiments, mutations range in size from a single DNA building block (DNA base) to a large segment of a chromosome. In certain embodiments, mutations can include missense mutations, frameshift mutations, duplications, insertions, nonsense mutation, deletions, and repeat expansions. In certain embodiments, a missense mutation is a change in one DNA base pair that results in the substitution of one amino acid for another in the protein made by a gene. In certain embodiments, a nonsense mutation is also a change in one DNA base pair. Instead of substituting one amino acid for another, however, the altered DNA sequence prematurely signals the cell to stop building a protein. In certain embodiments, an insertion changes the number of DNA bases in a gene by adding a piece of DNA. In certain embodiments, a deletion changes the number of DNA bases by removing a piece of DNA. In certain embodiments, small deletions can remove one or a few base pairs within a gene, while larger deletions can remove an entire gene or several neighboring genes. In certain embodiments, a duplication consists of a piece of DNA that is abnormally copied one or more times. In certain embodiments, frameshift mutations occur when the addition or loss of DNA bases changes a gene's reading frame. A reading frame consists of groups of 3 bases that each code for one amino acid. In certain embodiments, a frameshift mutation shifts the grouping of these bases and changes the code for amino acids. In certain embodiments, insertions, deletions, and duplications can all be frameshift mutations. In certain embodiments, a repeat expansion is another type of mutation. In certain embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In certain embodiments, a repeat expansion is a mutation that increases the number of times that the short DNA sequence is repeated.

As used herein, the term “sample” refers to a biological sample obtained or derived from a source of interest, as described herein. In certain embodiments, a source of interest comprises an organism, such as an animal or human. In certain embodiments, a biological sample is a biological tissue or fluid. Non-limiting examples of biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom, etc.

As used herein, the term “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested). In preferred embodiments, the subject is a human.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease condition, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

### Exemplary System Embodiments

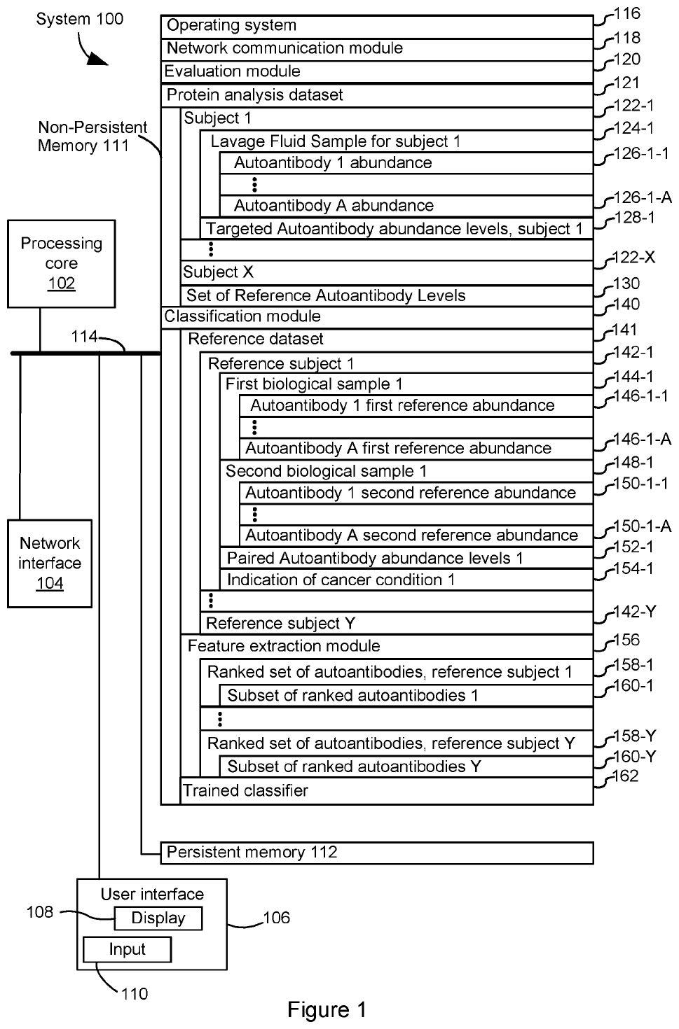

Details of an exemplary system are now described in conjunction with FIG. 1. FIG. 1 is a block diagram illustrating a system 100 in accordance with some implementations. The system 100 in some implementations includes at least one or more processing units CPU(s) 102 (also referred to as processors), one or more network interfaces 104, a display 106 having a user interface 108, an input device 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium, and stored thereon computer-executable executable instructions, which can be in the form of programs, modules, and data structures. In some implementations, the non-persistent memory 111 or alternatively the non-transitory computer readable storage medium stores the following programs, modules and data structures, or a subset thereof, sometimes in conjunction with the persistent memory 112:
an operating system 116, which includes procedures for handling various basic system services and for performing hardware-dependent tasks;
an optional network communication module (or instructions) 118 for connecting the system 100 with other devices and/or to a communication network;
an evaluation module 120 for evaluating a subject (e.g., subject 122-1, subject 122-2, . . . , and/or subject 122-X) for a stage of endometrial or ovarian cancer;
a protein analysis dataset 121 comprising, for each subject (e.g., subject 122-1), a plurality of antibody abundances (126-1-1, . . . 126-1-A) from a lavage fluid sample 124-1, and a set of protein abundance levels 128-1, and a set of reference protein abundance levels 130 (e.g., for filtering each plurality of protein abundances to obtain the corresponding set of targeted protein abundance levels for the respective subject); and
a classification module 140 for training a classifier to evaluate a subject for a stage of endometrial or ovarian cancer, comprising a reference dataset 141, a feature extraction module 156, and a trained classifier 162, where:
   the reference dataset 141 comprises, for each reference subject 142-1, 142-2, . . . 142-Y, a first biological sample (e.g., 144-1) and a second biological sample (e.g., 148-1), a set of paired protein abundance levels 152-1, and an indication of a disease (e.g., cancer) condition for the respective reference subject 154-1, where the first biological sample includes a first reference abundance for each protein in a plurality of proteins (e.g., 146-1-1, . . . 146-1-A), and the section biological sample includes a second reference abundance for each protein in the plurality of proteins (e.g., 150-1-1, . . . 150-1-A); and
   the feature extraction module 156 comprises a ranked set of proteins for each reference subject (e.g., 158-1, . . . 158-Y) and a subset of ranked proteins (160-1, . . . , 160-Y).

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements are stored in a computer system other than the system 100, that is addressable by the system 100 so that the system 100 may retrieve all or a portion of such data when needed

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items can be separate. Moreover, although FIG. 1 depicts certain data and modules in non-persistent 111 or persistent memory 112, it should be appreciated that these data and modules, or portion(s) thereof, may be stored in more than one memory. For example, in some embodiments, at least the evaluation module 120, the protein analysis dataset 121, and the classification module 140 are stored in a remote storage device that can be a part of a cloud-based infrastructure. In some embodiments, at least the protein analysis dataset 121 is stored on a cloud-based infrastructure. In some embodiments, the evaluation module 120 and the classification module 140 can also be stored in the remote storage device(s).

While an example of a system in accordance with the present disclosure has been disclosed with reference to FIG. 1, methods in accordance with the present disclosure are now detailed.

Classifiers

In some embodiments, the methods described herein use protein abundance values (also referred to herein as expression levels) to classify the state of a disorder, such as a gynecological disorder, in a subject. Generally, any classifier architecture can be trained for these purposes. Non-limiting examples of classifier types that can be used in conjunction with the methods described herein include a machine learning algorithm, molecular signature algorithm, a neural network algorithm, a support vector machine algorithm, a decision tree algorithm, an unsupervised clustering model algorithm, a supervised clustering model algorithm, or a regression model. In some embodiments, the trained classifier is binomial or multinomial.

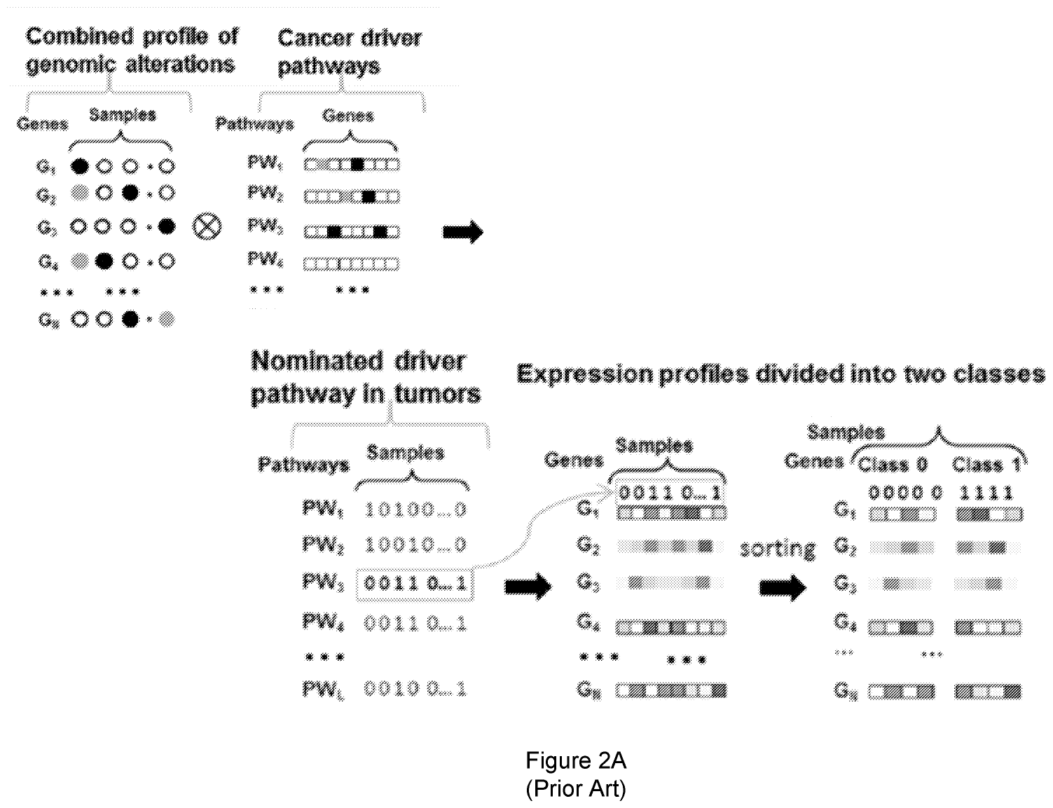

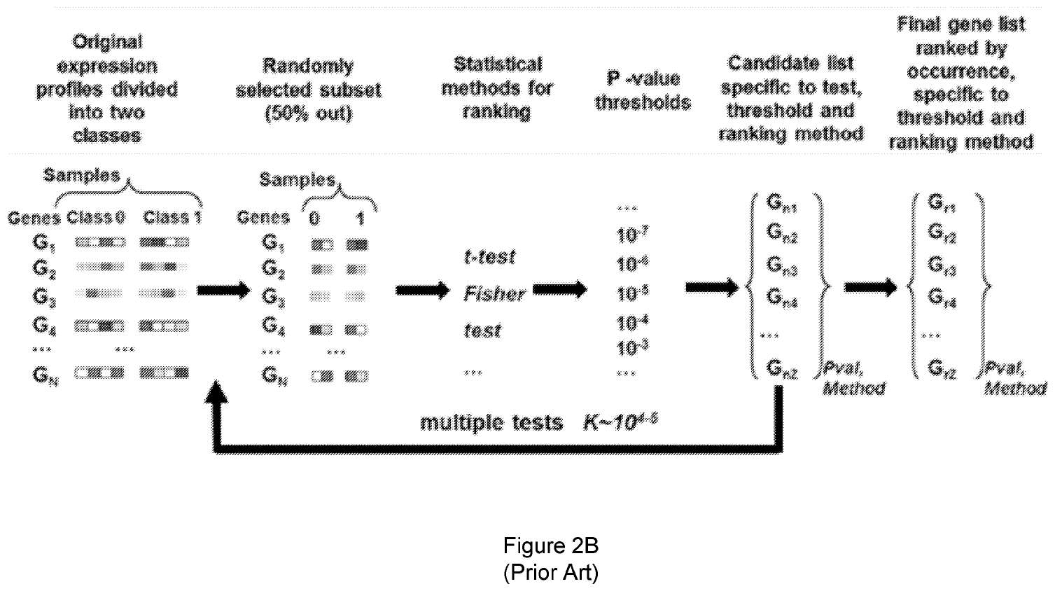

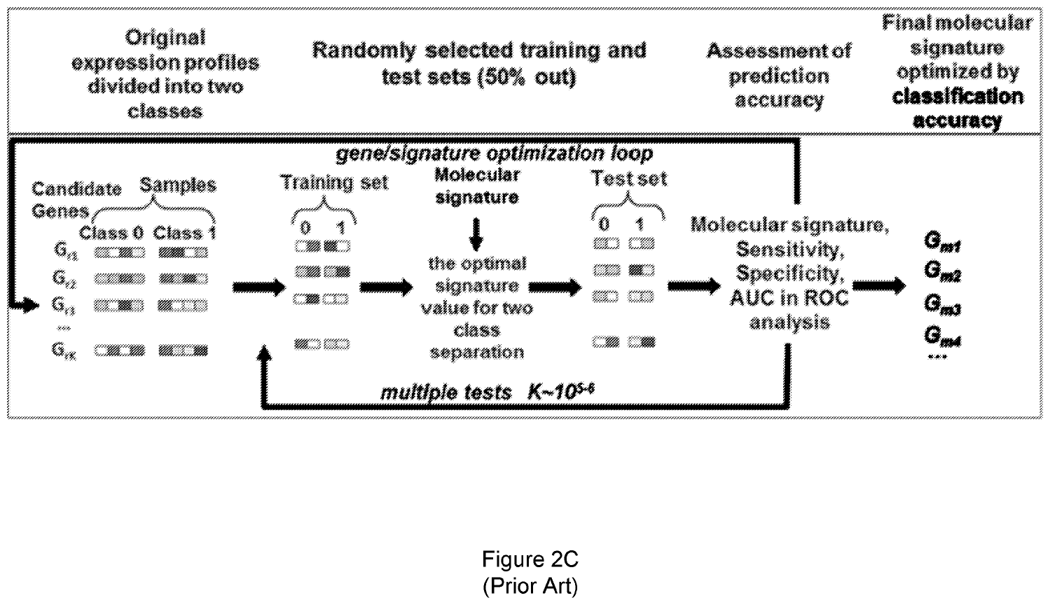

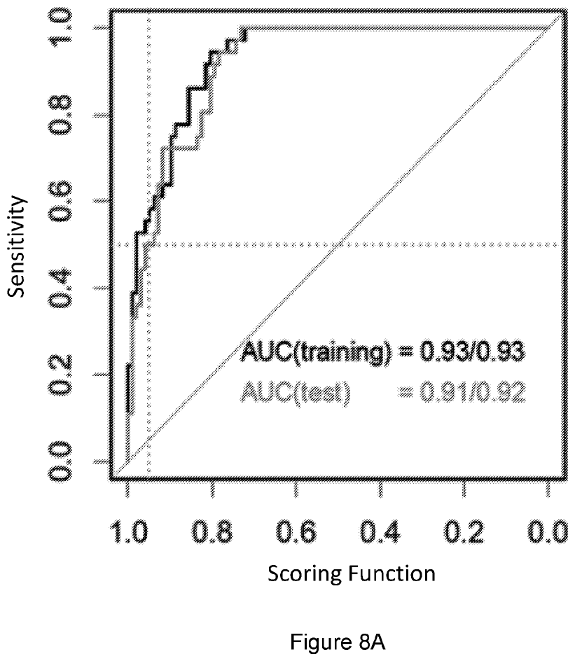

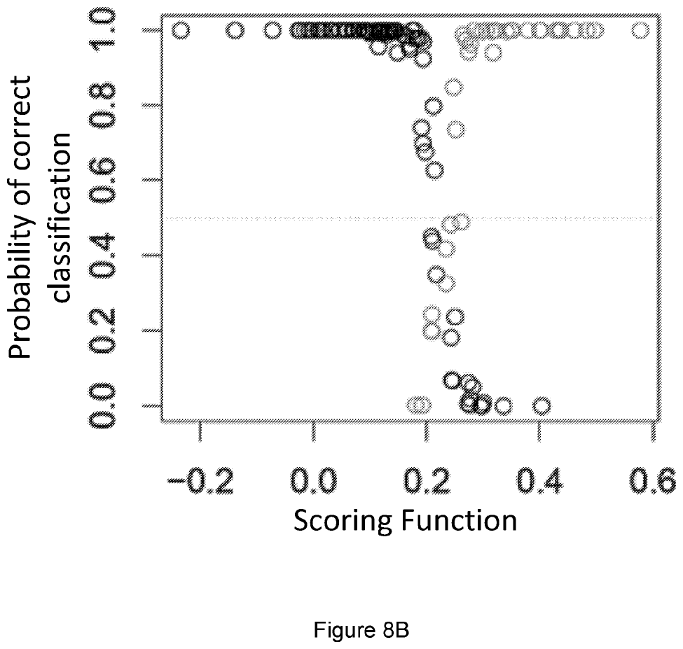

In some embodiments, the classifier includes a molecular signature model (MSM). See, Rykunov et al. et al. 2016 Nuc Acids Res 44(11), e110, the content of which is incorporated herein, by reference, in its entirety for all purposes. FIGS. 8A-8C illustrate an example of identifying molecular signatures with driver mutations (e.g., in accordance with MSM). As shown in FIG. 2A, in some embodiments, tumor molecular profiles from a plurality of subjects can be filtered using known driver alterations in molecular pathways, and different classes (e.g., for cancer vs. non-cancer or for two or more cancer conditions) of molecular expression profiles (e.g., molecular pathways with driver alterations) can be determined. FIG. 2B illustrates how potential molecular pathways and/or cell type signatures (e.g., the expression profile classes 1 and 0) can, in some embodiments, be ranked by occurrence (e.g., genes with expression levels that fall below predetermined p-value thresholds are discarded). In some embodiments, the overall set of molecular expression profiles can be subdivided (e.g., by randomly selecting 50% of the samples) into training and test datasets, and then the genes can be ranked using a t-test or a Fisher test (e.g., using the difference between the two expression profile classes 1 and 0). In some embodiments, this subdivision can be repeated one or more times (e.g., for 10⁴ or 10⁵ times) for determining a list of candidate molecular pathways and/or cell type signatures. These candidate molecular pathways and/or cell type signatures can be further evaluated for accuracy (e.g., the arithmetic mean of sensitivity and specificity) to determine a molecular signature comprising a set of gene expressions (e.g., average expression levels), for example as outlined in FIG. 2C.

Example logistic regression algorithms are disclosed in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, that can serve as the classifier for the instant methods are disclosed in See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

Support vector machine (SVM) algorithms that can serve as the classifier for the instant methods are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary-labeled data training set with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Decision trees (e.g., random forest, boosted trees) that can serve as the classifier for the instant methods are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can serve as the classifier for the instant methods is a classification and regression tree (CART). Other specific decision tree algorithms that can serve as the classifier for the instant methods include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U. C. Berkeley, September 1999, which is hereby incorporated by reference in its entirety.

In some embodiments, the methods described herein input protein abundance features into a machine learning algorithm to determine a prediction. The output of the machine learning algorithm may be a prediction of whether the subject has a disease, such as endometrial cancer, ovarian cancer, or breast cancer. Predictions of other diseases may also be possible in other embodiments. The use of measurements of protein abundance levels to predict diseases is not limited to only predicting a certain type of cancer. Also, the prediction may take various forms, depending on the machine learning algorithm. For example, the prediction may be a probability or likelihood that the subject has a disease condition. The prediction may also be a classification, such as a binary classification predicting the subject has a disease condition or does not have the disease condition, or multi-class output predicting what kinds of diseases the subject may have among a selection of diseases (e.g., a selection of various types of cancer).

In various embodiments, a wide variety of machine learning techniques may be used. Examples of which include different forms of unsupervised learning, clustering, supervised learning such as random forest classifiers, support vector machine (SVM) such as kernel SVMs, gradient boosting, linear regression, logistic regression, and other forms of regressions. Deep learning techniques such as neural networks, including recurrent neural networks (RNN) and long short-term memory networks (LSTM), may also be used. Customized machine learning techniques, such as molecular signature model (MSM), may also be used.

In a certain embodiment, a machine learning model may include certain layers, nodes, and/or coefficients. The machine learning model may be associated with an objective function, which generates a metric value that describes the objective goal of the training process. For example, the training may intend to reduce the error rate of the model by reducing the output value of the objective function, which may be called a loss function. Other forms of objective functions may also be used, particularly for unsupervised learning models whose error rates are not easily determined due to the lack of labels.

In one embodiment, a supervised learning technique is used. Patients with known disease conditions may be classified into two groups, which may be referred to as a positive training set (patients with the disease condition) and a negative training set (patients without the disease condition). In some supervised learning techniques, the objective function of the machine learning algorithm may be the training error rate in predicting the patients in the two training sets. For example, the objective function may be cross-entropy loss. In another embodiment, an unsupervised learning technique is used and the patients used in training are not labeled with disease condition. Various unsupervised learning technique such as clustering may be used. In yet another embodiment, the machine learning model may be semi-supervised.

Taking an example of a neural network as the machine learning model, training of the CNN may include forward propagation and backpropagation. A neural network may include an input layer, an output layer, and one or more intermediate layers that may be referred to as hidden layers. Each layer may include one or more nodes, which may be fully or partially connected to other nodes in adjacent layers. In forward propagation, the neural network performs computation in the forward direction based on outputs of a preceding layer. The operation of a node may be defined by one or more functions. The functions that define the operation of a node may include various computation operations such as convolution of data with one or more kernels, recurrent loop in RNN, various gates in LSTM, etc. The functions may also include an activation function that adjusts the weight of the output of the node. Nodes in different layers may be associated with different functions.

Each of the functions in a machine learning model may be associated with different coefficients that are adjustable during training. In addition, some of the nodes in a neural network each may also be associated with an activation function that decides the weight of the output of the node in forward propagation. Common activation functions may include step functions, linear functions, sigmoid functions, hyperbolic tangent functions (tanh), and rectified linear unit functions (ReLU). The data of a patient in the training set may be converted to a feature vector in a manner described above. After a feature vector is inputted into the neural network and passes through a neural network in the forward propagation, the results may be compared to the training label of the patient to determine the neural network's performance. The process of prediction may be repeated for other patients in the training sets to compute the value of the objective function in a particular training round. In turn, the neural network performs backpropagation by using coordinate descent such as stochastic coordinate descent (SGD) to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. A trained model may be used to predict the disease condition of a new subject.

While the training is described using a neural network as an example, a similar training process may be used for other suitable machine learning algorithms. In training a machine learning algorithm, various regularization techniques and cross-validation techniques may be used to reduce the chance of over-fitting the algorithm.

Classifier Features

In some embodiments of the methods described herein, e.g., method 700, classifiers use protein abundance data to determine values for each of a set of protein abundance features, which are used in the classification process. As described herein, in some embodiments, the protein abundance features are abundance values for proteins, logs of the protein abundance values, or a normalized protein abundance value thereof. For instance, in some embodiments, a normalization technique is applied to the protein abundance values or logs thereof, such as scaling to a range, clipping, log scaling, or determining a z-score.

However, systemic errors and batch effects were encountered when the protein abundance values, or logs thereof, were used to train a classifier. To define diagnostic biomarkers that are less sensitive to systematic errors and batch effects, a method was developed where the biomarkers and related classification functions can be applicable to a single sample. One way to satisfy this condition, i.e. minimization to a single sample, is to normalize all biomarkers by a computationally-derived “housekeeper” marker. Conventionally, a specific and pre-defined “housekeeping” gene, RNA sequence or protein, depending on the type of analyte being measured, is selected as the internal control. All subsequent measurements are then compared to that single housekeeper. However this method is non-trivial and can suffer from a number of issues including the necessity of a constant and non-zero expression value across all samples for that housekeeper and the ability to identify a priori such a housekeeper for the type of experiment being conducted. See, for example, Eisenberg E, Levanon E Y. Human housekeeping genes, revisited. Trends Genet. 2013 October; 29(10):569-74, Turabelidze A, Guo S, DiPietro L A. Importance of housekeeping gene selection for accurate reverse transcription-quantitative polymerase chain reaction in a wound healing model. Wound Repair Regen. 2010 September-October; 18(5):460-6, Tunbridge E M, Eastwood S L, Harrison P J. Changed relative to what? Housekeeping genes and normalization strategies in human brain gene expression studies. Biol Psychiatry. 2011 Jan. 15; 69(2):173-9, Wang Z, Lyu Z, Pan L, Zeng G, Randhawa P. Defining housekeeping genes suitable for RNA-seq analysis of the human allograft kidney biopsy tissue. BMC Med Genomics. 2019 Jun. 17; 12(1):86, Wi§ niewski J R, Mann M. A Proteomics Approach to the Protein Normalization Problem: Selection of Unvarying Proteins for MS-Based Proteomics and Western Blotting. J Proteome Res. 2016 Jul. 1; 15(7):2321-6, Kloubert V, Rink L. Selection of an inadequate housekeeping gene leads to misinterpretation of target gene expression in zinc deficiency and zinc supplementation models. J Trace Elem Med Biol. 2019 December; 56:192-197, and Chapman J R, Waldenström J. With Reference to Reference Genes: A Systematic Review of Endogenous Controls in Gene Expression Studies. PLoS One. 2015 Nov. 10; 10(11):e0141853, the contents of which are incorporated by reference herein, in their entireties, for all purposes.

In addition, given experimental differences in technical measurements, the “housekeeping” role may not be effectively translatable across different batches of test samples or testing under different conditions. See, for example, Asiabi P, Ambroise J, Giachini C, Coccia M E, Bearzatto B, Chiti M C, Dolmans M M, Amorim C A. Assessing and validating housekeeping genes in normal, cancerous, and polycystic human ovaries. J Assist Reprod Genet. 2020 October; 37(10):2545-2553, Maremanda K P, Sundar I K, Li D, Rahman I. Age-dependent assessment of genes involved in cellular senescence, telomere and mitochondrial pathways in human lung tissue of smokers, COPD and IPF: Associations with SARS-CoV-2 COVID-19 ACE2-TMPRSS2-Furin-DPP4 axis. medRxiv [Preprint], 2020 Jun. 16:2020.06.14.20129957, Bettencourt J W, McLaury A R, Limberg A K, Vargas-Hernandez J S, Bayram B, Owen A R, Berry D J, Sanchez-Sotelo J, Morrey M E, van Wijnen A J, Abdel M P. Total Protein Staining is Superior to Classical or Tissue-Specific Protein Staining for Standardization of Protein Biomarkers in Heterogeneous Tissue Samples. Gene Rep. 2020 June; 19:100641, Rai S N, Qian C, Pan J, McClain M, Eichenberger M R, McClain C J, Galandiuk S. Statistical Issues and Group Classification in Plasma MicroRNA Studies With Data Application. Evol Bioinform Online. 2020 Apr. 14; 16:1176934320913338, Dos Santos K C G, Desgagne-Penix I, Germain H. Custom selected reference genes outperform pre-defined reference genes in transcriptomic analysis. BMC Genomics. 2020 Jan. 10; 21(1):35, Zhang B, Wu X, Liu J, Song L, Song Q, Wang L, Yuan D, Wu Z. β-Actin: Not a Suitable Internal Control of Hepatic Fibrosis Caused by Schistosoma japonicum. Front Microbiol. 2019 Jan. 31; 10:66, Veres-Szekely A, Pap D, Sziksz E, Javorszky E, Rokonay R, Lippai R, Tory K, Fekete A, Tulassay T, Szabó A J, Vannay A. Selective measurement of a smooth muscle actin: why β-actin cannot be used as a housekeeping gene when tissue fibrosis occurs. BMC Mol Biol. 2017 Apr. 27; 18(1):12, and Wi§ niewski J R, Mann M. A Proteomics Approach to the Protein Normalization Problem: Selection of Unvarying Proteins for MS-Based Proteomics and Western Blotting. J Proteome Res. 2016 Jul. 1; 15(7):2321-6, the contents of which are incorporated by reference herein, in their entireties, for all purposes.

In some embodiments of a computationally-derived “housekeeper” marker method, the normalized profiles are defined as follows: Q′ᵢₛ=Qᵢₛ^{□}/Nᵢₛ^{□}, where Qᵢₛ^{□} is the original abundance level (e.g. expression level amount detected) of a marker i in a sample s, and Nₛ^{□} is an abundance level of a housekeeper marker in a sample s. In this manner, it is possible to search for a “computationally-derived housekeeper” by testing as all candidate housekeepers (with non-zero abundance levels in all samples) and determine the one, which makes possible the most accurate classification.

Alternatively, in some embodiments, a biomarker is defined as a comparison, e.g., ratio, of expression values: Q′ᵢₛ=Qᵢₛ^{□}/Nᵢₛ^{□}. This approach implies that the biological invariants (and differences) are determined by ratios of biological features rather than by absolute values of the features. In this iteration the biological features are molecular signals, which can include but are not limited to gene expression levels, protein abundance, epigenetic and posttranslational modifications, etc. This also means that the essential biological differences are more strongly associated with molecular signal ratios rather than with the absolute values of signals.

In support of this second iteration, biomarkers as ratios of expression values, we introduced and tested “pairwise biomarkers” defined as the differences between logarithms of abundance levels of all pairs of proteins. While this example uses proteins, we believe any dataset wherein differences between pairs can be defined, proteomic (mass spectroscopy data, proteins, peptide fragments), genomic (RNA expression levels, microbiome data), etc. can be so converted.

Thus, and in the examples provided below, for M proteins and, respectively, M*(M−1)/2 unique pairs of proteins, the differences between logs of abundance levels in each of the samples were computed and those pairwise differences were themselves used as biomarkers. Because the total number of unique pairs in protein profiles is large ˜15*10⁶, some statistically significant associations can be produced by random rather than by true underlying biological associations. To control for the possibility of random associations, in some embodiments, additional tests are performed with randomized distributions of diagnosis labels in sample cohorts to assess probabilities of random occurrence of statistically significant associations between pairwise biomarkers and diagnoses. Based on this test, in some embodiments, a P value threshold (Mann-Whitney-Wilcoxon test) is determined to sort out non-diagnosis related pairwise biomarkers produced by random. For instance, in some of the examples provided below, the results were obtained using statistical thresholds set at Pv<10⁻⁶⁻⁷, which excludes or minimizes random associations between pairwise biomarkers and diagnoses.

Advantageously, the statistical differentiation between protein profiles of patients of different diagnoses increases when pairwise biomarkers—ratios of logs of protein abundances are used. Further, using pairwise biomarkers makes possible classification of protein profiles with clinically relevant accuracy.

For measurements such as protein abundance levels, the measurement value may be used directly as a feature. The measurement value may also be mapped to another value based on one or more formulas (e.g., linear scaling or non-linear mapping). For traits such as genotypes, phenotypes, medical records of the subject that may not be naturally represented by a number, the trait may be converted to a number or a scale. For example, a presence or absence of a phenotype may be represented by a binary number. A dominant allele or a recessive allele may also be represented by a binary number. Some traits may be represented by a scale. The trait represented by a number may likewise be mapped to another value based on one or more formulas. Other features are also possible. For example, the features can be any suitable values that can be used in differentiating samples—demographic characteristics (e.g. Age, BMI, . . . ), results of blood test, average abundances of proteins representing molecular pathways from different pathway database; assessments of activities of molecular pathways; scoring functions derived from subnetworks of proteins and many other things which can used. Any quantitative assessments that can be deduced from protein abundances. These numerical assessments may be treated as features. In one embodiment, the set of numerical values may include only measurements of the targeted protein abundance levels that are obtained from the liquid biological sample, e.g., blood plasma or uterine lavage sample. In another embodiment, the set of numerical values may additionally include measurements of the targeted protein abundance levels that are obtained from a second biological sample. In yet another embodiment, the set of numerical values may further include values derived from other sources such as the subject's genotype data, morphometric data, and other suitable identifiable traits.

Example Feature Selection and Classifier Training Methodology

In some embodiments, the methods described herein rely upon a two-step computational protocol, including (i) use of a statistical algorithm for determining candidate features that are associated with pathway-specific genomic alterations and (ii) use of a machine learning algorithm for determining the optimal weights of combinations of candidate features to derive scoring functions—a signature for predicting key driver alterations in major cancer pathways. One embodiment of this process is described in Rykunov et al. et al. 2016 Nuc Acids Res 44(11), e110, which is incorporated herein by reference, in its entirety, for all purposes.

In some embodiments, the methods include selecting a ranked list of biomarkers by (1) defining a list of biomarkers, e.g., pairwise biomarkers as a difference between logarithms of given molecular signals (e.g. gene expression levels, protein abundances, etc. . . . ), and (2) using a boosting technique to rank the biomarkers, e.g., pairwise biomarkers. In order to boost, an original data set is repeatedly divided by random into, e.g., equal, training and test sets, and biomarkers, e.g., pairwise biomarkers, differentially distributed between two classes in both sets are been identified and ranked both by statistical power (P value) and by occurrence. For more information on this boosting technique see, for example, Rykunov et al. et al. 2016 Nuc Acids Res 44(11), e110.

Next, a classifier is identified by running classification tests and determining the optimal classification signature. In some embodiments, the algorithm takes as input a ranked list of candidate biomarkers (e.g., from steps 1 and 2, described above) and a dataset of molecular profiles. All possible sets of biomarkers are been tested by adding biomarkers singly and in succession. For each of the biomarker sets (typically, from 2 to 35) a dataset of molecular profiles is divided into two classes (e.g. cancer/benign, or Polyps/no Polyps). A classification function that optimizes the separation between given diagnostic classes is then computed as a weighted sum of biomarker levels, where weights are computed analytically using correlations between pairs of selected biomarkers. The training set is used to determine biomarker weights and optimal classification thresholds to be tested in the independent test set. For each samples of test set, the scoring function is computed using sample biomarker's values and weights determined in training set; then classifications is made based on the threshold of training set. The overall accuracy of classification is assess in multiple classification tests where half of a given dataset is used as training set and another half is used as test set. Thus, for each set of a ranked list of candidate biomarkers and each samples, the probability of correct classification and average scoring were computed in multiple classification tests. These values were then used for computation of overall classification accuracies assessed by area under receiver operating curve (AUC) both for averaged classification scores and for probabilities. Based on the obtained AUC values, the final list of biomarkers, their weights, and classification threshold is determined. For more information on this classifier identification technique see, for example, Rykunov et al. et al. 2016 Nuc Acids Res 44(11), e110.

Evaluating a Subject for a State of a Gynecologic Disorder

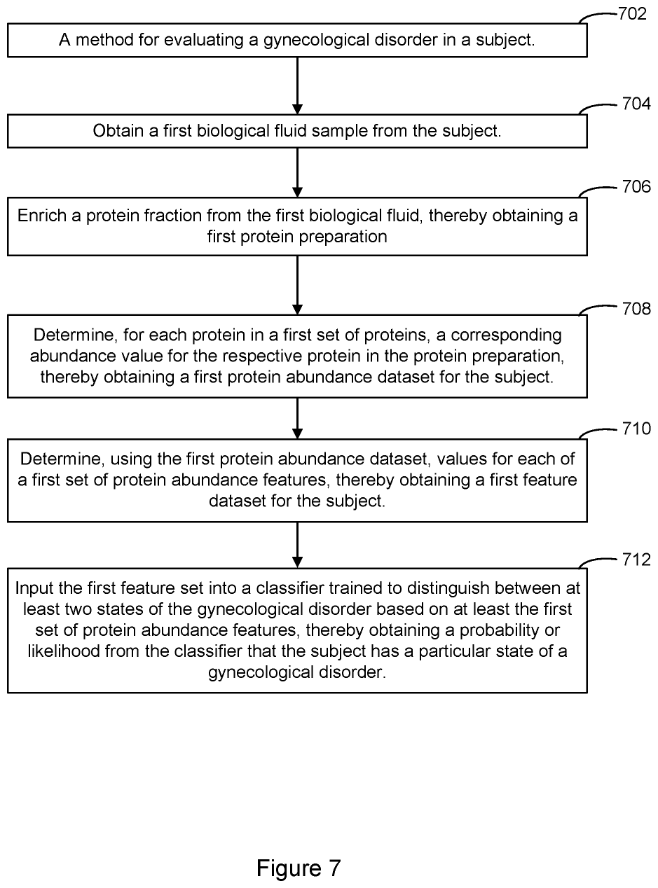

FIG. 7 example method 700 for evaluating a gynecological disorder (also referred to herein as an ovarian or uterine disease) in a subject using protein biomarkers found in a biological fluid sample, e.g., a blood plasma or uterine lavage fluid, from the subject.

Referring to block 1402 of FIG. 14, a method is provided for evaluating an ovarian or uterine disease condition in a subject. In some embodiments, the ovarian or uterine disease condition is an ovarian cancer or an endometrial cancer. In some embodiments, the ovarian or uterine disease condition is adenomyosis, endometrial polyps, leiomyoma, or endometriosis (e.g., complex atypical hyperplasia and/or an atrophic endometrium and/or an endometrial thickening).

In some embodiments, the method evaluates a subject for a disease condition. In some such embodiments, the disease condition comprises a non-cancerous condition. In some embodiments, the non-cancerous condition is endometriosis, tuberculosis, fungal infections, or bacterial pneumonias. See Radha et al. et al. 2014 J Cytol. 31(3), 136-138. In some embodiments, the non-cancerous condition is pericoronitis, hematemesis, ulcerative colitis, ulcer, osteoarthritis, sinusitis, or other conditions known in the art.

In some such embodiments, the disease condition comprises a pre-cancerous or cancer condition. A pre-cancerous disease condition involves abnormal cells that are at an increased risk of developing into cancer. In some embodiments, the cancer condition comprises endometrial cancer, ovarian cancer, cervical cancer, uterine sarcoma, vaginal cancer, vulvar cancer, gestational trophoblastic disease, or other reproductive cancer. In some embodiments, the cancer condition comprises breast cancer, esophageal cancer, lung cancer, renal cancer, colorectal cancer, nasopharyngeal cancer, lymphoma, or any other cancer condition known in the art.

In some embodiments, the stage of endometrial cancer comprises stage 0 endometrial cancer (e.g., complex atypical hyperplasia), stage IA endometrial cancer, stage IB endometrial cancer, stage II endometrial cancer, stage III endometrial cancer, or stage IV endometrial cancer. In some embodiments, the stage of ovarian cancer comprises stage 0 ovarian cancer, stage IA ovarian cancer, stage IB ovarian cancer, stage II ovarian cancer, stage III ovarian cancer, or stage IV ovarian cancer.

In some embodiments, the subject is asymptomatic for endometrial cancer. In some embodiments, the subject is asymptomatic for ovarian and/or endometrial cancer. In some embodiments, subjects are asymptomatic for endometrial cancer but do exhibit complex atypical hyperplasia (CAH). This is a pre-cancerous state (e.g., equivalent to stage 0 endometrial cancer) that is associated with an approximately 40% increased risk of a subject developing endometrial cancer. See e.g., Suh-Burgmann et al. et al. 2009 Obstetrics and Gynecology 114(3), 523-529. In some embodiments, the subject is symptomatic for ovarian and/or endometrial cancer. In some embodiments, a subject is from a population with an increased risk for ovarian and/or endometrial cancer. In some embodiments, the increased risk is that the subject has Lynch syndrome, the subject is obese, the subject has family history of ovarian and/or endometrial cancer, the subject has a BRCA mutation, and/or the subject is over a predetermined age—e.g., where the predetermined age is at least 40, at least 45, at least 50, at least 55, at least 60, at least 65, or at least 70 years of age). In some embodiments, the subject is asymptomatic. In some embodiments, the subject is experiencing pelvic pain, abnormal bleeding, or infertility.

In some embodiments, a subject is concurrently evaluated for a stage of an additional cancer condition distinct from ovarian and endometrial cancer. In some embodiments, another cancer condition is selected from the group consisting of lung cancer, prostate cancer, colorectal cancer, renal cancer, cancer of the esophagus, cervical cancer, bladder cancer, gastric cancer, nasopharyngeal cancer, or a combination thereof.

In some embodiments, the gynecological disorder is an ovarian cancer or an endometrial cancer. In some embodiments, the gynecological disorder is adenomyosis, endometrial polyps, leiomyoma, or endometriosis (e.g., complex atypical hyperplasia and/or an atrophic endometrium and/or an endometrial thickening). In some embodiments, the subject is asymptomatic. In some embodiments, the subject is experiencing pelvic pain, abnormal bleeding, or infertility.

Referring to block 704, the evaluation method proceeds by obtaining a first biological fluid sample, e.g., a blood plasma or uterine lavage fluid, from the subject. In some embodiments, a uterine lavage fluid is collected from the subject via hysteroscopy combined with curettage. In some embodiments, uterine lavage fluid is collected from the subject via uterine washings.

In some embodiments, a second biological fluid is collected from the subject. In some embodiments, the second biological fluid is a lavage fluid. In some embodiments, the lavage fluid sample is a bronchoalveolar lavage fluid sample, a gastric lavage fluid sample, a ductal lavage fluid sample, a nasal irrigation sample, a peritoneal lavage fluid sample, a peritoneal lavage fluid sample, an arthroscopic lavage fluid sample, or ear lavage fluid sample. In some embodiments, the second biological fluid is blood or a fraction thereof, such as a blood plasma fraction.

In some embodiments, a body cavity from which the lavage fluid sample is collected determines which type(s) of cancer said lavage fluid sample is assayed for (e.g., bladder cancer, oral cancer, lung cancer, gastrointestinal cancer, endometrial, and/or ovarian). In some such embodiments, the method further evaluates the subject for a stage of bladder cancer, a stage of oral cancer, a stage of lung cancer, a stage of gastrointestinal cancer, a stage of endometrial cancer, and/or a stage of ovarian cancer, respectively.

In some embodiments, the first biological fluid sample includes blood, bone marrow, urine, ascites, sputum, saliva, urine, cerebrospinal fluid, peritoneal fluid, pleural fluid, feces, lymph fluid, gynecological fluids, skin swab, vaginal swab, oral swab, nasal swab, feces, uterine lavage fluid, bladder lavage fluid, oral rinse, or lung washings. In some embodiments, the first biological fluid sample is a uterine lavage fluid.

Referring to block 706, the evaluation method proceeds by enriching a protein fraction from the first biological fluid, thereby obtaining a first protein preparation.

Referring to block 708, the evaluation method proceeds by determining for each protein in a first set of proteins, a corresponding abundance value for the respective protein in the protein preparation. The method thereby includes obtaining a first protein abundance dataset for the subject.

Table 1 lists features found to be informative for distinguishing between (i) the presence of polyps and (ii) no polyps in a protein preparation from uterine lavage fluid. Each feature represents a ratio of (i) the log of the abundance of the first listed protein, to (ii) the log of the abundance of the second listed protein. For instance, feature MACF1_SNRPF refers to a comparison (e.g., a ratio) of (i) the log abundance of human MACF1 protein in a biological fluid sample, to (ii) the log abundance of human SNRPF protein in the biological fluid sample. Accordingly, in some embodiments, the first set of proteins includes human MACF1 protein. Similarly, in some embodiments, the first set of proteins includes human SNRPF protein. Likewise, in some embodiments, the first set of proteins includes human MACF1 protein and human SNRPF protein.

In some embodiments, the first set of proteins includes at least 3 proteins listed in Table 1. In some embodiments, the first set of proteins includes at least 5 proteins listed in Table 1. In some embodiments, the first set of proteins includes at least 10 proteins listed in Table 1. In some embodiments, the first set of proteins includes at least 25 proteins listed in Table 1. In some embodiments, the first set of proteins includes at least 50 proteins listed in Table 1. In some embodiments, the first set of proteins includes at least 2, 3, 4, 5, 6, 7, 8, 9, 10, 11, 12, 13, 14, 15, 16, 17, 18, 19, 20, 25, 30, 35, 40, 45, 50, or more proteins listed in Table 1.

TABLE 1
Example features found to be informative for distinguishing between
(i) the presence of polyps and (ii) no polyps in a protein
preparation from uterine lavage fluid. Each feature represents a
ratio of (i) the log of the abundance of the first listed protein,
to (ii) the log of the abundance of the second listed protein.
Example Features

MACF1_{——}SNRPF
GSPT1_{——}SNRPF
C5_{——}SNRPF
C5_{——}FUS
HNRNPL_{——}RNF40
HNRNPL_{——}RANBP3
C5_{——}HNRNPL
H2BC14_{——}MACF1
IGFALS_{——}SNRPF
COL14A1_{——}SNRPF
ARPC1A_{——}SNRPF
BLMH_{——}SNRPF
PRDX6_{——}SNRPF
LBP_{——}NACA
LBP_{——}SYNCRIP
HNRNPL_{——}RAN
HNRNPD_{——}RAN
RPL23A_{——}SNRPF
KRT10_{——}YBX1
RNF40_{——}YBX1
APOA1_{——}SNRPF
FLG_{——}SNRPF
CHMP1B_{——}SNRPF
PRDX6_{——}SYNCRIP
PGK1_{——}SNRPF
ACO2_{——}NPM1
MXRA5_{——}SNRPF
HNRNPL_{——}PRDX2
SNRPF_{——}SPTBN1
IGFALS_{——}RCC2
HSPA8_{——}SNRPF
EVPL_{——}SNRPF
HRNR_{——}NPM1
RAN_{——}SNRPF
KRT10_{——}NPM1
HNRNPD_{——}MACF1
KRT2_{——}SNRPF
GSPT1_{——}NCL
GGT1_{——}HNRNPL
ACTR3_{——}SNRPF
GSPT1_{——}H2BC14
APOA4_{——}SNRPF
HNRNPL_{——}RPN1
EIF5_{——}HNRNPD
C5_{——}HNRNPD
CLTC_{——}FUS
H2BC14_{——}IGFALS
HNRNPL_{——}MME
RPL23A_{——}RPS19
SNRPF_{——}TLN1
DSG1_{——}SNRPF
IGHG4_{——}SNRPF
HNRNPL_{——}MACF1
H4C9_{——}MACF1
HNRNPL_{——}PRPF8
HNRNPD_{——}RNF40
ACP1_{——}HNRNPL
HBG1_{——}NPM1
HNRNPL_{——}SLC2A1
EVPL_{——}HNRNPL
C8A_{——}SNRPF
HBA2_{——}SNRPF
BROX_{——}SNRPF
GTF2I_{——}HRNR
HNRNPL_{——}IGKV2D-40
BLVRB_{——}FUS
IGHV3-64D_{——}MTDH
HBG1_{——}HNRNPD
KRT10_{——}SNRPF
HAL_{——}PSAT1
IDH1_{——}NPM1
CFL1_{——}HNRNPL
HBG1_{——}HNRNPL
ACO2_{——}GTF2I
RANBP3_{——}SNRPF
HNRNPL_{——}RPS27A
CRIP2_{——}KRT10
PRDX2_{——}SNRPF
BLVRB_{——}HNRNPD
MYOF_{——}RNF40
SERPINA3_{——}SNRPF
C8G_{——}SNRPF
HNRNPL_{——}PSMD2
H2BC14_{——}PRDX6
C4B_2_{——}SNRPF
KRT13_{——}SSRP1
C5_{——}MYOF
KRT10_{——}MTDH
HBG1_{——}SNRPF
APOA4_{——}HMGB3
HBB_{——}SNRPF
SNRPF_{——}TALDO1
H4C9_{——}IGFALS
RPL23A_{——}SYNCRIP
MTDH_{——}PRDX2
BLVRA_{——}SNRPF
SLC2A1_{——}SNRPF
CNDP2_{——}HNRNPL
HBG1_{——}YBX1
APOA1_{——}MYOF
H2BC14_{——}HSPA8
HAL_{——}RPL29
PCBP2_{——}VPS4B
ARG1_{——}STIM1
H2AC6_{——}LGALS3
ACP1_{——}SNRPF
DDX42_{——}HNRNPL
SSRP1_{——}TFF3
RPS27A_{——}SNRPF
H2BC14_{——}SERPINA1
KRT9_{——}MTDH
GSPT1_{——}H4C9
HBD_{——}SNRPF
GTF2I_{——}IDH1
MTDH_{——}RANBP3
GLG1_{——}RNF40
H2BC14_{——}VWA5A
IGHV3-64D_{——}YBX1
KHSRP_{——}MXRA5
CLTC_{——}HNRNPL
BLVRA_{——}H2BC14
HAL_{——}RPS9
GLG1_{——}IGHV3-64D
RANBP3_{——}SRP14
H2BC14_{——}TXN
FUS_{——}UPF1
H2BC14_{——}HAL
GSPT1_{——}KHSRP
IGKV2D-40_{——}STIM1
C2_{——}H2BC14
EIF4H_{——}IGHV3-64D
KRT3_{——}NUCKS1
RAN_{——}RBM8A
ARPC2_{——}RANBP3
CPB2_{——}FUS
H2BC14_{——}RPL23A
GSPT1_{——}PPP1CB
PPP1CB_{——}RANBP3
IGLV1-51_{——}YBX1
CTTN_{——}HBG1
GSPT1_{——}LRRC59
C5_{——}JPT2
IGHG4_{——}LRRC59
EIF2S1_{——}IGHV3-64D
H2BC14_{——}LARS1
MAPK1_{——}YBX1
ARCN1_{——}NUCKS1
RANBP3_{——}SND1

Table 2 lists features found to be informative for distinguishing between (i) the presence of polyps and (ii) no polyps in a protein preparation from blood plasma. Each feature represents a ratio of (i) the log of the abundance of the first listed protein, to (ii) the log of the abundance of the second listed protein. For instance, feature AGT_RASGRP2 refers to a comparison (e.g., a ratio) of (i) the log abundance of human AGT protein in a biological fluid sample, to (ii) the log abundance of human RASGRP2 protein in the biological fluid sample. Accordingly, in some embodiments, the first set of proteins includes human AGT protein. Similarly, in some embodiments, the first set of proteins includes human RASGRP2 protein. Likewise, in some embodiments, the first set of proteins includes human AGT protein and human RASGRP2 protein.

In some embodiments, the first set of proteins includes at least 3 proteins listed in Table 2. In some embodiments, the first set of proteins includes at least 5 proteins listed in Table 2. In some embodiments, the first set of proteins includes at least 10 proteins listed in Table 2. In some embodiments, the first set of proteins includes at least 25 proteins listed in Table 2. In some embodiments, the first set of proteins includes at least 50 proteins listed in Table 2. In some embodiments, the first set of proteins includes at least 2, 3, 4, 5, 6, 7, 8, 9, 10, 11, 12, 13, 14, 15, 16, 17, 18, 19, 20, 25, 30, 35, 40, 45, 50, or more proteins listed in Table 2.

TABLE 2
Example features found to be informative for distinguishing between
(i) the presence of polyps and (ii) no polyps in a protein
preparation from blood plasma. Each feature represents a ratio of
(i) the log of the abundance of the first listed protein, to (ii)
the log of the abundance of the second listed protein.
Example Features

AGT_{——}RASGRP2
ATP5F1B_{——}P4HB
HSP90AB1_{——}TTR
PSTPIP2_{——}TTR
CNDP1_{——}PSTPIP2
APOA4_{——}PSMB8
CPN2_{——}RASGRP2
AK1_{——}APOA4
ALB_{——}PSTPIP2
ALB_{——}HSP90AB1
PSMB8_{——}TTR
FLOT1_{——}KRT14
CNDP1_{——}PSMB8
KRT14_{——}RASGRP2
APOA4_{——}PGK1
GC_{——}PSMB8
AK1_{——}CNDP1
AGT_{——}LDHB
APOA4_{——}HSP90AB1
RASGRP2_{——}TTR
APOA4_{——}PSTPIP2
CPN2_{——}PDLIM5
PSMD2_{——}TTR
CPN2_{——}HSP90AB1
PSTPIP2_{——}SERPINA1
APOA4_{——}PNP
APOC1_{——}PSTPIP2
CAB39_{——}TTR
P4HB_{——}TTR
CPN2_{——}FLOT1
APOA4_{——}SEPTIN7
NFIB_{——}RASGRP2
APOA4_{——}CAB39
CNDP1_{——}SEPTIN5
AGT_{——}ARF6
GC_{——}PSTPIP2
CPN2_{——}P4HB
CNDP1_{——}PSMB6
CNDP1_{——}TUBB
DLST_{——}TTR
CNDP1_{——}TXNDC5
CNDP1_{——}CORO1C
TTR_{——}YWHAE
PRDX6_{——}TTR
APOA4_{——}ARHGDIA
PGK1_{——}TTR
CALD1_{——}CNDP1
CNDP1_{——}PNP
LDHB_{——}NFIB
APOA4_{——}OPA1
OPA1_{——}TTR
CNDP1_{——}TNS1
CPN2_{——}PRDX6
CNDP1_{——}SEPTIN7
ATP5PO_{——}CNDP1
APOA4_{——}SEPTIN5
HSP90AB1_{——}THBS1
HSP90AB1_{——}NFIB
CNDP1_{——}RAB1B
APOA4_{——}FGA
ATP2A3_{——}P4HB
CCT6A_{——}CPN2
APOC3_{——}RASGRP2
KRT14_{——}MTPN
PSMB6_{——}TTR
CNDP1_{——}SEC31A
CPN2_{——}TXNL1
CAPN1_{——}TTR
HSP90AB1_{——}IGHM
C1QA_{——}RAB1B
CNDP1_{——}EIF4E
SEPTIN7_{——}TTR
CNDP1_{——}TLN2
DLST_{——}GC
PNP_{——}TTR
NFIB_{——}PRDX6
CNDP1_{——}HSP90AB1
SEPTIN5_{——}TTR
HSP90AB1_{——}YARS1
TTR_{——}WDR44
CHMP4B_{——}CNDP1
FGA_{——}PRSS1
SYTL4_{——}TTR
APOA4_{——}EIF4E
CPN2_{——}GNA13
APOA4_{——}PDIA4
APOA4_{——}RAB1B
PACSIN2_{——}PRSS1
TTR_{——}WBP2
ATP6V1E1_{——}KRT14
HSP90AB1_{——}ITGA5
TARS1_{——}TTR
GGCT_{——}RASGRP2
ANXA5_{——}CNDP1
ATP2A3_{——}HSP90AB1
RASGRP2_{——}YARS1
C1QB_{——}PSTPIP2
GC_{——}YWHAE
ATP5PB_{——}TTR
TPM1_{——}TTR
CPN2_{——}RENBP
RAB1B_{——}TTR
ARHGDIA_{——}TTR
ATP6V1E1_{——}NFIB
LYN_{——}TTR
ARF6_{——}NFIB
MLEC_{——}TTR
SERPINA1_{——}STAT3
C1QB_{——}PNP
ALB_{——}PNP
CLIC1_{——}CPN2
ENDOD1_{——}P4HB
ENO1_{——}P4HB
CPN2_{——}PSMA6
APOC1_{——}PNP
PECAM1_{——}TTR
ORM1_{——}SYNE1
AGT_{——}RAB7A
CNDP1_{——}WBP2
ALB_{——}YWHAE
GSN_{——}PRDX6
PNP_{——}PPIF
CNDP1_{——}SERPIND1
CNDP1_{——}TXNL1
ALDOA_{——}APOA4
F5_{——}PNP
GSN_{——}RASGRP2
NEXN_{——}PNP
CPN2_{——}PSMB8
OPA1_{——}PRSS1
ARHGAP45_{——}SYNE1
MAP1A_{——}PNP
APOH_{——}PACSIN2
CPN2_{——}PRDX1
CNDP1_{——}WDR44
CPN2_{——}PLTP
CCT6A_{——}UNC45A
PSTPIP2_{——}VTN
HSD17B4_{——}PNP
CD109_{——}PNP
SRC_{——}TTR
CAPZA1_{——}CNDP1
IGHM_{——}RASGRP2
ATP5F1B_{——}PRDX6

Table 3 lists features found to be informative for distinguishing between (i) the presence of endometrial cancer and (ii) a benign phenotype in a protein preparation from uterine lavage fluid. Each feature represents a ratio of (i) the log of the abundance of the first listed protein, to (ii) the log of the abundance of the second listed protein. For instance, feature APPL1_YBX1 refers to a comparison (e.g., a ratio) of (i) the log abundance of human APPL1 protein in a biological fluid sample, to (ii) the log abundance of human YBX1 protein in the biological fluid sample. Accordingly, in some embodiments, the first set of proteins includes human APPL1 protein. Similarly, in some embodiments, the first set of proteins includes human YBX1 protein. Likewise, in some embodiments, the first set of proteins includes human APPL1 protein and human YBX1 protein.

In some embodiments, the first set of proteins includes at least 3 proteins listed in Table 3. In some embodiments, the first set of proteins includes at least 5 proteins listed in Table 3. In some embodiments, the first set of proteins includes at least 10 proteins listed in Table 3. In some embodiments, the first set of proteins includes at least 25 proteins listed in Table 3. In some embodiments, the first set of proteins includes at least 50 proteins listed in Table 3. In some embodiments, the first set of proteins includes at least 2, 3, 4, 5, 6, 7, 8, 9, 10, 11, 12, 13, 14, 15, 16, 17, 18, 19, 20, 25, 30, 35, 40, 45, 50, or more proteins listed in Table 3.

TABLE 3
Example features found to be informative for distinguishing between
(i) the presence of endometrial cancer and (ii) a benign phenotype
in a protein preparation from uterine lavage fluid. Each feature
represents a ratio of (i) the log of the abundance of the first listed
protein, to (ii) the log of the abundance of the second listed protein.
Example Features

APPL1_{——}YBX1
XRCC6_{——}YBX1
MYH14_{——}YBX1
NCL_{——}NFIB
IGLV1-51_{——}YBX1
HMGB3_{——}XRCC6
PSMD2_{——}YBX1
TUBB4B_{——}YBX1
SLK_{——}YBX1
RAN_{——}YBX1
FERMT3_{——}SYNCRIP
HMGB3_{——}MUC5B
ARHGAP1_{——}HMGB3
FERMT3_{——}HMGB3
HDGFL3_{——}RTCB
HMGB3_{——}NFIB
IGHV3-30_{——}PARK7
KRT6A_{——}YBX1
APPL1_{——}HMGB3
HMGB3_{——}KRT6A
HMGB3_{——}TUBB4B
IGKC_{——}YBX1
JCHAIN_{——}YBX1
PROM1_{——}YBX1
ARHGAP1_{——}SYNCRIP
CBR1_{——}HMGB3
HMGB3_{——}PTPN11
SUPT16H_{——}TUBB4B
SYNCRIP_{——}XRCC6
APPL1_{——}NPM1
NCL_{——}NPEPPS
BPIFB1_{——}HMGB3
FLG_{——}SNRPF
RNF40_{——}YBX1
C4BPB_{——}H4C9
CP_{——}YBX1
H4C9_{——}NFIB
HMGB3_{——}IGHM
H2BC14_{——}HSPA8
IGKV4-1_{——}YBX1
PIGR_{——}YBX1
H4C9_{——}NPEPPS
HMGB3_{——}HSPA8
HMGB3_{——}KRT2
NPEPPS_{——}YBX1
ERO1A_{——}EWSR1
IGHG3_{——}YBX1
IGHV3-64D_{——}YBX1
IGLV3-21_{——}YBX1
SYNCRIP_{——}THYN1
H4C9_{——}PROM1
HMGB3_{——}MCTS1
IGHV3-30_{——}YBX1
PFKL_{——}S100A14
SCYL2_{——}YBX1
HMGB3_{——}IGLV3-21
HMGB3_{——}KRT5
HMGB3_{——}NPEPPS
MUC5B_{——}YBX1
ACP1_{——}COPB2
CACYBP_{——}HMGB3
CP_{——}HMGB3
FERMT3_{——}YBX1
HMGB3_{——}MACF1
AGT_{——}HMGB3
APPL1_{——}RPL6
ARCN1_{——}SUPT16H
C4BPB_{——}HMGB3
DENR_{——}PFKM
FLG_{——}NCL
HMGB3_{——}HRNR
ACP1_{——}RPL6
DSP_{——}HMGB3
HMGB3_{——}TSG101
HMGB3_{——}VPS4B
STAT6_{——}YBX1
FLG_{——}SYNCRIP
H2BC14_{——}NFIB
H2BC14_{——}NPEPPS
HMGB3_{——}IGHV3-64D
HMGB3_{——}RNF40
NFIB_{——}YBX1
NPM1_{——}USP5
AGT_{——}H2BC14
BPIFB1_{——}H2BC14
CAB39_{——}H2BC14
DENR_{——}RNF40
FBP1_{——}RPL6
FERMT3_{——}RPL6
FLG_{——}SNRPA
GLUL_{——}YBX1
HMGB3_{——}S100A14
PTPN11_{——}SYNCRIP
SUPT16H_{——}XRCC6
ACO2_{——}SNRPF
APPL1_{——}DENR
CAB39_{——}HMGB3
DNPEP_{——}RPL6
FCGBP_{——}HMGB3
H2BC14_{——}LGALS3
H2BC14_{——}MUC5B
HMGB3_{——}KLK11
HMGB3_{——}PRDX6
HMGB3_{——}PROM1
JCHAIN_{——}RPL6
NCL_{——}PRDX6
NPEPPS_{——}SNRPA
RPL6_{——}THYN1
SNX2_{——}SUPT16H
SNX2_{——}SYNCRIP
SYNCRIP_{——}TKFC
AGT_{——}NUCKS1
APPL1_{——}SYNCRIP
BPIFB1_{——}YBX1
H2BC14_{——}IGLV3-21
H2BC14_{——}PIGR
HMGB3_{——}LGALS3
HMGB3_{——}THYN1
ACTN1_{——}YBX1
ATP5F1B_{——}SNRPA
COPB2_{——}EVPL
COPB2_{——}NPEPPS
EWSR1_{——}RNF40
HMGB3_{——}KRT77
HMGB3_{——}SERPINB3
HNRNPAB_{——}PPP2R2A
IGHV3-30_{——}PRG4
IGHV3-72_{——}YBX1
MVP_{——}RNF40
APEX1_{——}JCHAIN
C4BPB_{——}H2BC14
C4BPB_{——}RPSA
CP_{——}H2BC14
CP_{——}H4C9
FERMT3_{——}SUPT16H
GLUL_{——}HMGB3
GSTO1_{——}YBX1
HMGB3_{——}IVL
HMGB3_{——}JCHAIN
HMGB3_{——}KRT14
JCHAIN_{——}NCL
KLK11_{——}YBX1
KRT77_{——}NCL
NPEPPS_{——}RPL6
PRDX6_{——}RPL6
SUPT16H_{——}THYN1
SYNCRIP_{——}TUBB4B
CP_{——}NCL
FERMT3_{——}HNRNPR
FLG_{——}HMGB3
H2BC14_{——}PROM1
HMGB3_{——}ITIH1
HMGB3_{——}RPL23A
IGHAI_{——}YBX1
IGHV3-64D_{——}RPL6
KRT77_{——}YBX1
ACO2_{——}H2BC14
BPIFB1_{——}SNRPA
CAB39_{——}H4C9
CAB39_{——}SNRPA
CLU_{——}YBX1
GNG12_{——}SUPT16H
H2BC14_{——}JCHAIN
H4C9_{——}IGHV3-64D
HMGB3_{——}PRDX2
IGHM_{——}YBX1
MUC5B_{——}NCL
ATP6V1A_{——}SUPT16H
CFI_{——}YBX1
EVPL_{——}RPL6
FBP1_{——}HMGB3
H2BC14_{——}IGHV3-64D
H2BC14_{——}KRT16
IGKC_{——}PARK7
MME_{——}RPL6
NCL_{——}PROM1
NPM1_{——}RNF40
SPTBN1_{——}YBX1
VPS4B_{——}YBX1
APPL1_{——}RDX
BPIFB1_{——}H4C9
CBX3_{——}RNF40
COPB2_{——}VPS4B
FCGBP_{——}H2BC14
GSTK1_{——}SUPT16H
H4C9_{——}IGLV3-21
HMGB3_{——}PSMD2
IGKV3-15_{——}YBX1
IGLV3-21_{——}NCL
KARS1_{——}NPEPPS
STAT1_{——}YBX1
GSTK1_{——}NUCKS1
HMGB3_{——}PRRC2A
HMGB3_{——}RPA1
HMGB3_{——}VWA5A
PPL_{——}SUPT16H
PSMA6_{——}YBX1
RPL6_{——}SNX2
APPL1_{——}SNRPF
ARHGAP1_{——}SUPT16H
CHMP1B_{——}HMGB3
CP_{——}RPL6
FBP1_{——}MANF
H2BC14_{——}IGHV3-72
H2BC14_{——}TSG101
HSPA8_{——}SNRPA
IGHV3-30_{——}KLKB1
PARK7_{——}PIGR
RNF40_{——}RPL6
SF3B1_{——}YBX1
AARS1_{——}YBX1
CFI_{——}HMGB3
COPB2_{——}IGHV3-64D
EVPL_{——}YBX1
FKBP5_{——}YBX1
HMGB3_{——}JUP
HMGB3_{——}PGD
PRDX2_{——}YBX1
PRDX6_{——}SYNCRIP
RNF40_{——}THBS1
SYNE2_{——}YBX1
APEX1_{——}PRDX6
ARCN1_{——}SYNCRIP
FLNB_{——}YBX1
H2BC14_{——}IGHG4
H4C9_{——}RNF40
HMGB3_{——}PIGR
HNRNPD_{——}RNF40
HNRNPR_{——}PRDX6
IGHV3-64D_{——}RPS9
IGHV3-64D_{——}SUPT16H
NPEPPS_{——}SUPT16H
NPM1_{——}PROM1
PROM1_{——}RDX
COL1A1_{——}IGLV3-21
DENR_{——}VPS4B
FLG_{——}YBX1
HMGB3_{——}IGHG4
HMGB3_{——}IGKC
IGHV3-30_{——}QSOX1
IGLV1-47_{——}YBX1
NPEPPS_{——}SYNCRIP
PSMD2_{——}SUPT16H
CBR1_{——}H2BC14
FBP1_{——}NACA
HRNR_{——}NCL
IGHV6-1_{——}YBX1
LSP1_{——}RNF40
NPM1_{——}PRDX6
S100A14_{——}SUPT16H
SERPINB3_{——}SUPT16H
ACP1_{——}CBX3
CAB39_{——}HNRNPAB
CFI_{——}NPM1
DENR_{——}RAN
GLUL_{——}SUPT16H
HGD_{——}RNF40
IGHG2_{——}YBX1
IGHV3-30_{——}NPM1
C4BPB_{——}RCC2
CAB39_{——}SUPT16H
DENR_{——}IGHV3-30
DNPEP_{——}HMGB3
H2BC14_{——}SERPINB3
HNRNPR_{——}RNF40
IGHM_{——}NCL
PIGR_{——}RPL6
ESD_{——}H2BC14
EVPL_{——}HMGB3
HMGB3_{——}RAN
IGHV3-49_{——}YBX1
NPEPPS_{——}RCC2
PARK7_{——}STAT6
APEX1_{——}IGHV3-64D
CAB39_{——}SYNCRIP
DENR_{——}S100A14
H2BC14_{——}IGHG2
HMGB3_{——}IGHV3-30
HMGB3_{——}TPP2
NCL_{——}PIGR
CBX3_{——}MME
CHMP1B_{——}NCL
DENR_{——}GLUL
DNPEP_{——}LRRC59
FBP1_{——}YBX1
HRNR_{——}_{——}YBX1
KRT2_{——}YBX1
FLNA_{——}SYNCRIP
HMGB3_{——}UBA6
IGHV3-30_{——}RPL6
KLKB1_{——}RNF40
RBM8A_{——}RNF40
RNF40_{——}WARS1
RPS18_{——}S100A14
ACTC1_{——}RNF40
DNPEP_{——}SUPT16H
H2BC14_{——}RNF40
IGFBP2_{——}NPEPPS
IGHG2_{——}PARK7
IGHV3-64D_{——}RPSA
IGLV3-21_{——}PGM1
SERPINB3_{——}SNRPA
AZGP1_{——}YBX1
IGHV3-64D_{——}NPM1
IGLV3-21_{——}NPM1
RPL6_{——}SLC2A1
C1QC_{——}IGKC
FBP1_{——}LRRC59
HSPA1A_{——}RPL6
IGFBP2_{——}TSG101
IGHV3-64D_{——}NCL
BZW1_{——}IGHV3-64D
FLG_{——}HNRNPAB
FLNB_{——}HMGB3
SERPINB3_{——}SFN
C1QC_{——}RNF40
IGLV3-21_{——}RPL6
ACTR2_{——}YBX1
COPB2_{——}PRDX6
ERO1A_{——}H2BC14
IGHV3-64D_{——}KARS1
IGHV3-64D_{——}SUB1
C1QB_{——}IGLV3-21
HNRNPR_{——}IGHV3-64D
RPL23A_{——}YBX1
YBX1_{——}ZC3H18
IGHV3-30_{——}LSP1
HNRNPL_{——}RNF40
IGHV3-64D_{——}SRP9
IGHV3-64D_{——}SYNCRIP
NPM1_{——}PRDX2
APEX1_{——}HSPA8
C4BPB_{——}IGFBP2
DENR_{——}EVPL
H2BC14_{——}USP5
IGHV3-64D_{——}MANF
NPM1_{——}PIGR
HMGB3_{——}KCNB2
APEX1_{——}PSMD9
CBX3_{——}MYH14
RNF40_{——}TGFBI
PREX2_{——}THRAP3
CBR1_{——}NPM1
FLG_{——}SLTM
IGHV3-64D_{——}PSAT1
ARCN1_{——}HNRNPR
NACA_{——}PFKM
NCL_{——}PSMD9
RPL6_{——}RPN1
CSRP1_{——}SUPT16H
H2AC6_{——}NPEPPS
IGHG4_{——}YBX1
BPIFB1_{——}RPL10A
DNPEP_{——}RPS4X
HBG1_{——}SUB1
NAPRT_{——}RPL6
PSMD9_{——}SUB1
HBG1_{——}NPM1
MPO_{——}RNF40
FLG_{——}HDGFL3
RNF40_{——}SND1
AGT_{——}IGFBP2
H2BC14_{——}SRRM2
IDH2_{——}RPL15
APPL1_{——}RPL15
ALDH1A3_{——}THRAP3
CBR1_{——}MANF
CHMP1B_{——}HNRNPR
BCLAF1_{——}DSG1
PFKM_{——}RPS9

Table 4 lists features found to be informative for distinguishing between (i) the presence of endometrial cancer and (ii) a benign phenotype in a protein preparation from blood plasma. Each feature represents a ratio of (i) the log of the abundance of the first listed protein, to (ii) the log of the abundance of the second listed protein. For instance, feature ACTR2_SERPINA1 refers to a comparison (e.g., a ratio) of (i) the log abundance of human ACTR2 protein in a biological fluid sample, to (ii) the log abundance of human SERPINA1 protein in the biological fluid sample. Accordingly, in some embodiments, the first set of proteins includes human ACTR2 protein. Similarly, in some embodiments, the first set of proteins includes human SERPINA1 protein. Likewise, in some embodiments, the first set of proteins includes human ACTR2 protein and human SERPINA1 protein.

In some embodiments, the first set of proteins includes at least 3 proteins listed in Table 4. In some embodiments, the first set of proteins includes at least 5 proteins listed in Table 4. In some embodiments, the first set of proteins includes at least 10 proteins listed in Table 4. In some embodiments, the first set of proteins includes at least 25 proteins listed in Table 4. In some embodiments, the first set of proteins includes at least 50 proteins listed in Table 4. In some embodiments, the first set of proteins includes at least 2, 3, 4, 5, 6, 7, 8, 9, 10, 11, 12, 13, 14, 15, 16, 17, 18, 19, 20, 25, 30, 35, 40, 45, 50, or more proteins listed in Table 4.

TABLE 4
Example features found to be informative for distinguishing between
(i) the presence of endometrial cancer and (ii) a benign phenotype
in a protein preparation from blood plasma. Each feature represents
a ratio of (i) the log of the abundance of the first listed protein,
to (ii) the log of the abundance of the second listed protein.
A feature corresponding to a pair of biomarkers

ACTR2_{——}SERPINA1
ARPC1B_{——}SERPINA1
CFH_{——}SERPINA1
CYFIP1_{——}SERPINA1
FABP5_{——}SERPINA1
FASN_{——}SERPINA1
GNA13_{——}SERPINA1
PSMD2_{——}SERPINA1
PSTPIP2_{——}SERPINA1
PSTPIP2_{——}VTN
RAP1B_{——}SERPINA1
SERPINA1_{——}SRC
SERPINA1_{——}SYTL4
SERPINA1_{——}UTRN
SERPINA1_{——}YWHAE
SERPINA1_{——}YWHAZ
FLOT1_{——}SERPINA1
C3_{——}PSTPIP2
ACTR2_{——}VTN
RASGRP2_{——}SERPINA1
SRC_{——}VTN
HSPA5_{——}SERPINA1
GC_{——}YWHAE
C3_{——}SRC
RENBP_{——}SERPINA1
LYN_{——}SERPINA1
RAP2B_{——}SERPINA1
SERPINA1_{——}SNX2
PURB_{——}SERPINA1
MPP1_{——}SERPINA1
CAPN1_{——}SERPINA1
PSMB4_{——}SERPINA1
EHD1_{——}SERPINA1
PRDX6_{——}SERPINA1
SERPINA1_{——}SERPINB6
CCT4_{——}SERPINA1
HSP90AB1_{——}SERPINA1
SERPINA1_{——}TPP2
GNA13_{——}OBSCN
CSRP1_{——}SERPINA1
OBSCN_{——}RASGRP2
PRDX1_{——}SERPINA1
OBSCN_{——}PSMD2
P4HB_{——}SERPINA1
AMPD2_{——}SERPINA1
RHOG_{——}SERPINA1
GC_{——}GNA13
CNDP1_{——}YWHAZ
APOA4_{——}PSTPIP2
ANXA5_{——}SERPINA1
SELP_{——}SERPINA1
OBSCN_{——}PSTPIP2
PSMD2_{——}VTN
OBSCN_{——}YWHAE
CSRP1_{——}VTN
PRDX2_{——}YWHAZ

Referring to block 710, the evaluation method proceeds by determining, using the first protein abundance dataset, values for each of a first set of protein abundance features. The method thereby includes obtaining a first feature dataset for the subject. As described herein, in some embodiments, the protein abundance features are abundance values for proteins, logs of the protein abundance values, or a normalized protein abundance value thereof. For instance, in some embodiments, a normalization technique is applied to the protein abundance values or logs thereof, such as scaling to a range, clipping, log scaling, or determining a z-score.

In some embodiments, each respective feature in the first set of protein abundance features includes a normalized abundance value for a respective protein in the first set of proteins. In some embodiments, each respective feature in the first set of protein abundance features includes a comparison between an abundance value for a first respective protein in the first set of proteins and an abundance value for a second respective protein in the first set of proteins.

In some embodiments, the first set of protein abundance features includes at least 5 of the features listed in Table 1. In some embodiments, the first set of protein abundance features includes at least 10 of the features listed in Table 1. In some embodiments, the first set of protein abundance features includes at least 25 of the features listed in Table 1. In some embodiments, the first set of protein abundance features includes at least 50 of the features listed in Table 1. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, or all 148 of the features listed in Table 1.

In some embodiments, the first set of protein abundance features includes at least 5 of the features listed in Table 2. In some embodiments, the first set of protein abundance features includes at least 10 of the features listed in Table 2. In some embodiments, the first set of protein abundance features includes at least 25 of the features listed in Table 2. In some embodiments, the first set of protein abundance features includes at least 50 of the features listed in Table 2. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, or all 144 of the features listed in Table 2.

In some embodiments, the first set of protein abundance features includes at least 5 of the features listed in Table 3. In some embodiments, the first set of protein abundance features includes at least 10 of the features listed in Table 3. In some embodiments, the first set of protein abundance features includes at least 25 of the features listed in Table 3. In some embodiments, the first set of protein abundance features includes at least 50 of the features listed in Table 3. In some embodiments, the first set of protein abundance features includes at least 100 of the features listed in Table 3. In some embodiments, the first set of protein abundance features includes at least 200 of the features listed in Table 3. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 175, 200, 225, 250, 275, 300, 325, 350, or all 370 of the features listed in Table 3.

In some embodiments, the first set of protein abundance features includes at least 5 of the features listed in Table 4. In some embodiments, the first set of protein abundance features includes at least 10 of the features listed in Table 4. In some embodiments, the first set of protein abundance features includes at least 25 of the features listed in Table 4. In some embodiments, the first set of protein abundance features includes at least 50 of the features listed in Table 4. In some embodiments, the first set of protein abundance features includes at least 2, 3, 4, 5, 6, 7, 8, 9, 10, 11, 12, 13, 14, 15, 16, 17, 18, 19, 20, 25, 30, 35, 40, 45, 50, or all 56 of the features listed in Table 4.

In some embodiments, the first set of protein abundance features was determined by a feature selection method including steps of (1) defining a list of biomarkers, e.g., pairwise biomarkers as a difference between logarithms of given molecular signals (e.g. gene expression levels, protein abundances, etc.), and (2) using a boosting technique to rank the biomarkers, e.g., pairwise biomarkers. In some embodiments, the method further includes running a plurality of classification tests and determining the optimal classification signature. In some embodiments, the plurality of classification tests evaluate all possible combinations of biomarker sets having a range of features. For example, in some embodiments, the plurality of classification tests evaluate all possible combinations of biomarker sets having a minimum number of features and a maximum number of features. Generally, the skilled artisan will select the minimum number of features and maximum number of features based on the size of the master feature lists. In some embodiments, the minimum number of features is 2, 3, 4, 5, 6, 7, 8, 9, 10, 15, 20, or 25 features. In some embodiments, the maximum number of features is 25% of the total number of possible features, or 30%, 35%, 40%, 45%, 50%, 55%, 60%, 65%, 70%, 75%, 80%, 85%, 90%, 95%, 99%, or 100% of the total number of features.

Referring to block 712, the evaluation method inputs the first feature set into a classifier. The classifier is trained to distinguish between at least two states of the gynecological disorder based on at least the first set of protein abundance features. The method thereby includes obtaining a probability or likelihood from the classifier that the subject has a particular state of a gynecological disorder. As described above, many types of classifiers can be used in conjunction with the methods described herein.

In some embodiments, the classifier determines a disease profile Vₛ for the subject including a weighted sum Wₛ of the respective values for each of the first set of protein abundance features in the first feature dataset. Wₛ is calculated as:

Wₛ=Σᵢ₌₁^{m}(AᵢEᵢ),

where Eᵢ is a value of a respective protein abundance feature i, in the first feature dataset having m protein abundance features, determined for the first protein abundance dataset, and Aᵢ is a weight for protein abundance feature i.

In some embodiments, for each respective protein abundance features i in the first set of m protein abundance features, the weight A i is calculated as:

Aᵢ˜Dᵢ⁻¹Σⱼ₌₁^{k}([Cᵢⱼ]⁻¹Zⱼ),

where Dᵢ is the standard deviation of the value of the protein abundance feature i in a training set of biological fluid samples. The training set includes a first subset of biological fluid samples from training subjects having a first state of the gynecological disorder, and a second subset of biological fluid samples from training subjects having a second state of the gynecological disorder. Cᵢⱼ is a matrix of pairwise correlation between the values of protein abundance features i and j in the first training set, such that [Cᵢⱼ]⁻¹ is the reciprocal matrix of pairwise correlation, where k=m−1. Zⱼ is a z-score for the values of protein abundance feature j in the first training set. Zⱼ is calculated as:

$${Z}_{j} = \frac{{〈 {E}_{j} 〉}_{1} - {〈 {E}_{j} 〉}_{2}}{{D}_{j}} ,$$

where [symbol] Eⱼ [symbol] , is the average value of protein abundance feature j determined for the first subset of biological fluid samples, [symbol] Eⱼ [symbol] ₂ is the average value of protein abundance feature j determined for the second subset of biological fluid samples, and Dⱼ is the standard deviation of the values of protein abundance feature j determined for the training set of biological fluid samples.

In some embodiments, the classifier includes a molecular signature algorithm, a neural network algorithm, a support vector machine algorithm, a decision tree algorithm, an unsupervised clustering model algorithm, a supervised clustering model algorithm, or a regression model.

In some embodiments, the classifier was trained to distinguish between the at least two states of the gynecological disorder based on at least the values for each of a first set of protein abundance features and one or more secondary features for the subject.

In some embodiments, the gynecological disorder condition is an ovarian cancer or an endometrial cancer. In such embodiments, the one or more secondary features of the subject include two or more of the features selected from the group consisting of an age of the subject, a pregnancy history of the subject, a breastfeeding history of the subject, a BRCA1 genotype of the subject, a BRCA2 genotype of the subject, a breast cancer history of the subject, and a familial history of endometrial cancer, ovarian cancer, or breast cancer.

In some embodiments, the method further includes obtaining a second biological sample from the subject and determining a plurality of secondary features from the second biological sample. The method thereby includes obtaining a second feature dataset for the subject. The method also includes inputting the second feature dataset into the classifier.

In some embodiments, the second biological sample is a fluid biological sample. In some embodiments, the second biological sample is a blood plasma sample. In some embodiments, the second biological sample is a uterine lavage fluid sample. In some embodiments, the second biological fluid sample includes blood, bone marrow, urine, ascites, sputum, saliva, urine, cerebrospinal fluid, peritoneal fluid, pleural fluid, feces, lymph fluid, gynecological fluids, skin swab, vaginal swab, oral swab, nasal swab, feces, uterine lavage fluid, bladder lavage fluid, oral rinse, or lung washings.

In some embodiments, the classifier was trained to distinguish between (i) the presence of an ovarian cancer or uterine cancer and (ii) the absence of the ovarian cancer or the uterine cancer. The method further includes, when the probability or likelihood obtained from the classifier indicates that the subject has the ovarian cancer or the uterine cancer, administering a therapy for the ovarian cancer or the uterine cancer to the subject. The method also includes, when the probability or likelihood obtained from the classifier indicates that the subject does not have the ovarian cancer or the uterine cancer, forgoing administration of the therapy for the ovarian cancer or the uterine cancer to the subject.

In some embodiments, the classifier was trained to distinguish between (i) a first stage of an ovarian cancer or uterine cancer and (ii) a second stage of the ovarian cancer or the uterine cancer that is more advanced than the first stage of the ovarian cancer or the uterine cancer. The method further includes, when the probability or likelihood obtained from the classifier indicates that the subject has the first stage of the ovarian cancer or the uterine cancer, administering a first therapy for the ovarian cancer or the uterine cancer to the subject. The method also includes, when the probability or likelihood obtained from the classifier indicates that the subject has the first stage of the ovarian cancer or the uterine cancer, administering a second therapy for the ovarian cancer or the uterine cancer to the subject.

In some embodiments, the classifier was trained to distinguish between (i) the presence of adenomyosis, endometrial polyps, leiomyoma, or endometriosis and (ii) the absence of the adenomyosis, endometrial polyps, leiomyoma, or endometriosis. The method further includes, when the probability or likelihood obtained from the classifier indicates that the subject has the adenomyosis, endometrial polyps, leiomyoma, or endometriosis, administering a therapy for the adenomyosis, endometrial polyps, leiomyoma, or endometriosis to the subject. The method also includes, when the probability or likelihood obtained from the classifier indicates that the subject does not have the adenomyosis, endometrial polyps, leiomyoma, or endometriosis, forgoing administration of the therapy for the adenomyosis, endometrial polyps, leiomyoma, or endometriosis to the subject.

### EXAMPLES

### Example 1—Training of a Classifier to Distinguish Between the Presence of Endometrial Polyps and the Absence of Endometrial Polyps Based on Proteomics of Uterine Lavage Fluid

FIGS. 8A and 8B collectively illustrate the classification of patient samples derived from uterine lavage with regard to polyp diagnoses, in accordance with some embodiments of the present disclosure.

A classifier was trained against 36 protein profiles of polyp diagnosis vs 97 protein profiles of other diagnoses including 28 benign, 61 endometrial and 8 ovarian cancers determined from uterine lavage samples, e.g., using the master list of features listed in Table 1 above (e.g., pairwise comparisons between two protein abundances). For each possible feature set, the dataset was divided into two classes (e.g. Polyps/no Polyps). A classification function that optimizes the separation between given diagnostic classes was then computed as a weighted sum of biomarker levels, where weights are computed analytically using correlations between pairs of selected biomarkers. The training set was used to determine biomarker weights and optimal classification thresholds to be tested in the independent test set.

For each sampling of the test set, a scoring function was computed using sample biomarker's values and weights determined in the training set. Then, classifications was made based on the threshold of the training set. The overall accuracy of classification was assessed in multiple classification tests, where half of a given dataset is used as training set and another half is used as test set. Thus, for each set of a ranked list of candidate features and each sample, the probability of correct classification and average scoring were computed in multiple classification tests. These values were then used for computation of overall classification accuracies assessed by area under receiver operating curve (AUC) both for averaged classification scores and for probabilities.

Expression values of an optimal set of four protein abundance features, EIF5_HNRNPD, IGFALS_RCC2, H2AC6_LGALS3, and SNRPF_TLN1, were used to train a classifier. The classification accuracies were assessed by area under receiver operating curve (AUC), as illustrated in FIG. 8A. FIG. 8B illustrates averaged classification probabilities as functions of averaged scoring functions. The classification accuracy depends on scoring function and increases at the tails of the distribution. The high degree of consistency between AUCs is derived from scoring function and probability.

### Example 2—Training of a Classifier to Distinguish Between the Presence of Endometrial Polyps and the Absence of Endometrial Polyps Based on Proteomics of Blood Plasma

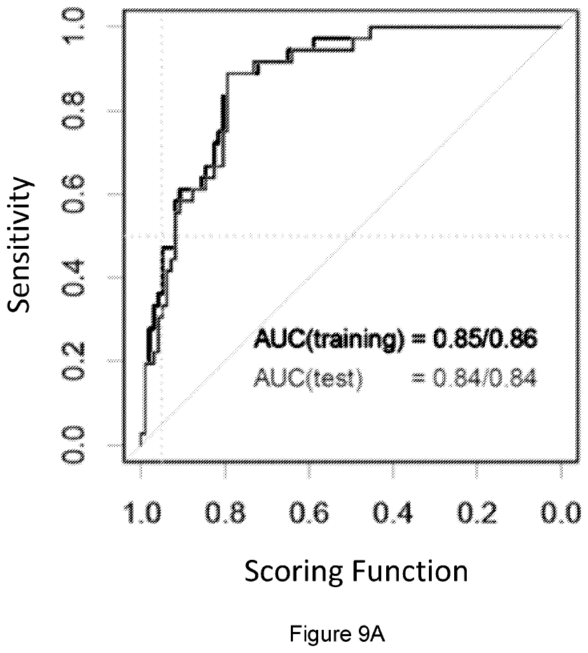

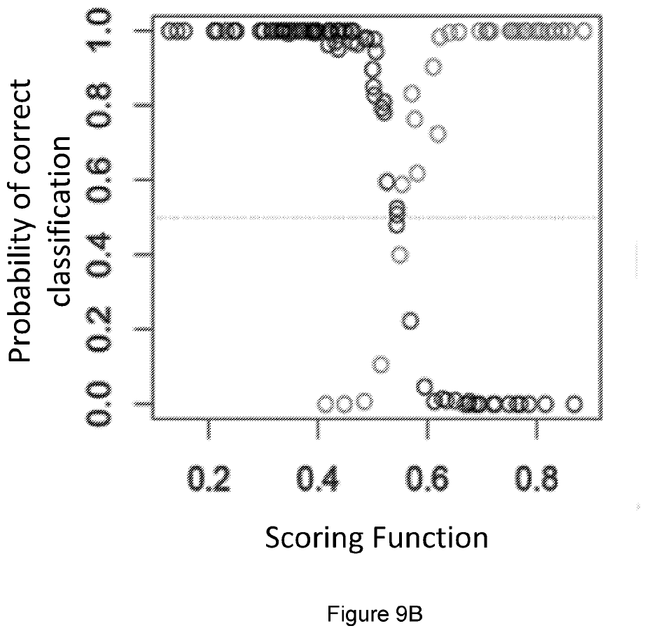

FIGS. 9A and 9B collectively illustrate the classification of patient samples derived from blood plasma with regard to polyp diagnoses, in accordance with some embodiments of the present disclosure.

A classifier was trained against 36 protein profiles of polyp diagnosis vs 97 protein profiles of other diagnoses including 28 benign, 61 endometrial and 8 ovarian cancers determined from blood plasma, e.g., using the master list of features listed in Table 2 above (e.g., pairwise comparisons between two protein abundances). For each possible feature set, the dataset was divided into two classes (e.g. Polyps/no Polyps). A classification function that optimizes the separation between given diagnostic classes was then computed as a weighted sum of biomarker levels, where weights are computed analytically using correlations between pairs of selected biomarkers. The training set was used to determine biomarker weights and optimal classification thresholds to be tested in the independent test set.

For each sampling of the test set, a scoring function was computed using sample biomarker's values and weights determined in the training set. Then, classifications was made based on the threshold of the training set. The overall accuracy of classification was assessed in multiple classification tests, where half of a given dataset is used as training set and another half is used as test set. Thus, for each set of a ranked list of candidate features and each sample, the probability of correct classification and average scoring were computed in multiple classification tests. These values were then used for computation of overall classification accuracies assessed by area under receiver operating curve (AUC) both for averaged classification scores and for probabilities.

Expression values of an optimal set of three protein abundance features, FLOT1_KRT14, APOA4_PGK1, and AGT_RASGRP2, were used to train a classifier. The classification accuracies were assessed by area under receiver operating curve (AUC), as illustrated in FIG. 9A. FIG. 9B illustrates averaged classification probabilities as functions of averaged scoring functions. The classification accuracy depends on scoring function and increases at the tails of the distribution. The high degree of consistency between AUCs is derived from scoring function and probability.

### Example 3—Training of a Classifier to Distinguish Between the Presence of Endometrial Polyps and Other Benign Diagnoses Based on Proteomics of Uterine Lavage Fluid

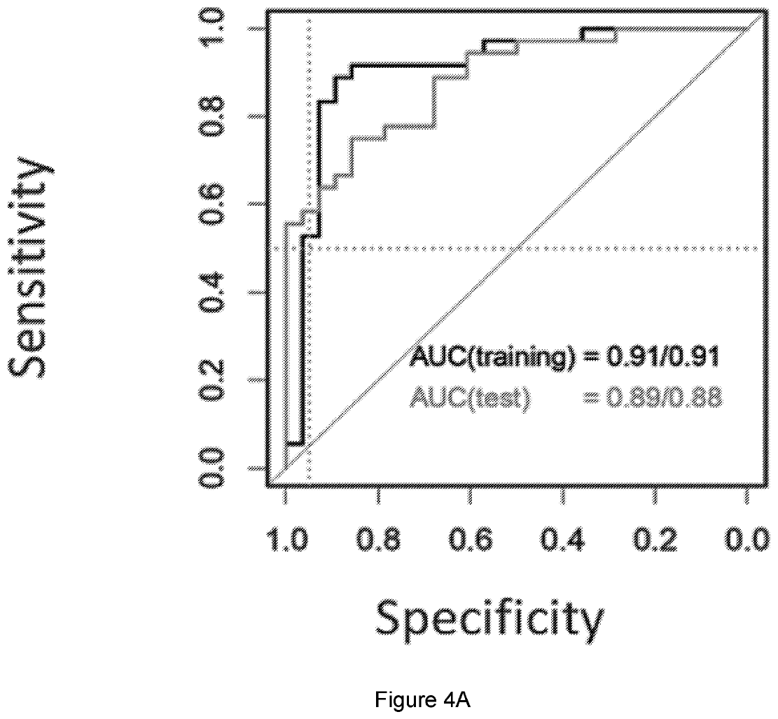

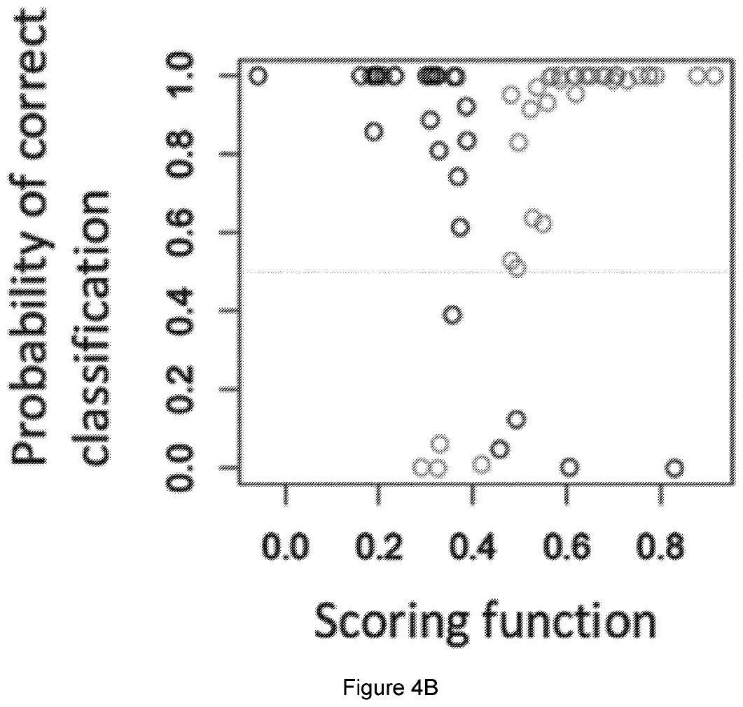

FIGS. 4A and 4B collectively illustrate the classification of patient samples derived from uterine lavage with regard to polyp diagnoses, in accordance with some embodiments of the present disclosure.

A classifier was trained against 36 protein profiles of polyp diagnosis vs 28 protein profiles of other benign diagnoses determined from uterine lavage samples using a master list of features, e.g., pairwise comparisons between two protein abundances. For each possible feature set, the dataset was divided into two classes (e.g. Polyps/no Polyps). A classification function that optimizes the separation between given diagnostic classes was then computed as a weighted sum of biomarker levels, where weights are computed analytically using correlations between pairs of selected biomarkers. The training set was used to determine biomarker weights and optimal classification thresholds to be tested in the independent test set.

For each sampling of the test set, a scoring function was computed using sample biomarker's values and weights determined in the training set. Then, classifications was made based on the threshold of the training set. The overall accuracy of classification was assessed in multiple classification tests, where half of a given dataset is used as training set and another half is used as test set. Thus, for each set of a ranked list of candidate features and each sample, the probability of correct classification and average scoring were computed in multiple classification tests. These values were then used for computation of overall classification accuracies assessed by area under receiver operating curve (AUC) both for averaged classification scores and for probabilities.

Expression values of an optimal set of three protein abundance features, EIF4H_LBP, FUS_UPF1, and APOA1_PAIP were used to train a classifier. The classification accuracies were assessed by area under receiver operating curve (AUC), as illustrated in FIG. 4A. FIG. 4C illustrates averaged classification probabilities as functions of averaged scoring functions. The classification accuracy depends on scoring function and increases at the tails of the distribution. The high degree of consistency between AUCs is derived from scoring function and probability.

### Example 4—Training of a Classifier to Distinguish Between the Presence of Endometrial Polyps and Other Benign Diagnoses Based on Proteomics of Blood Plasma

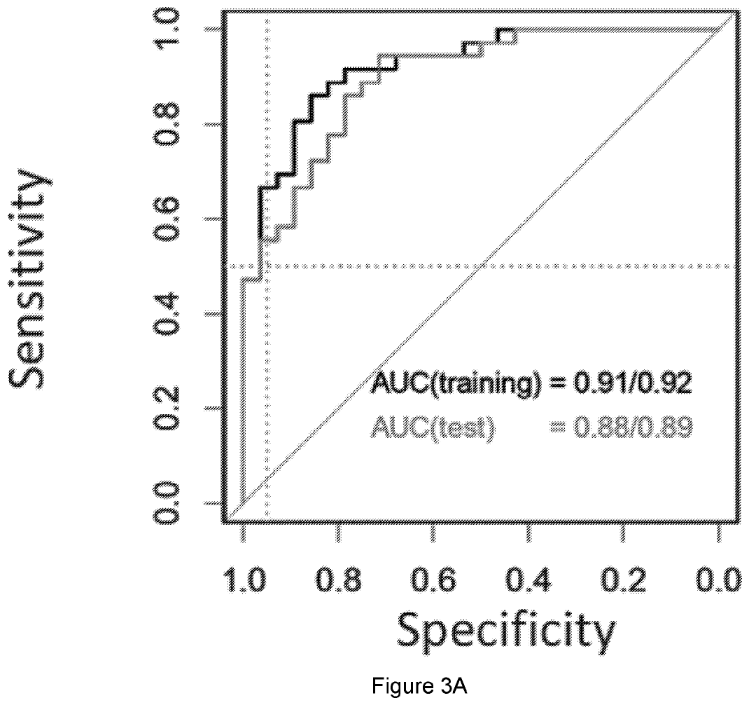

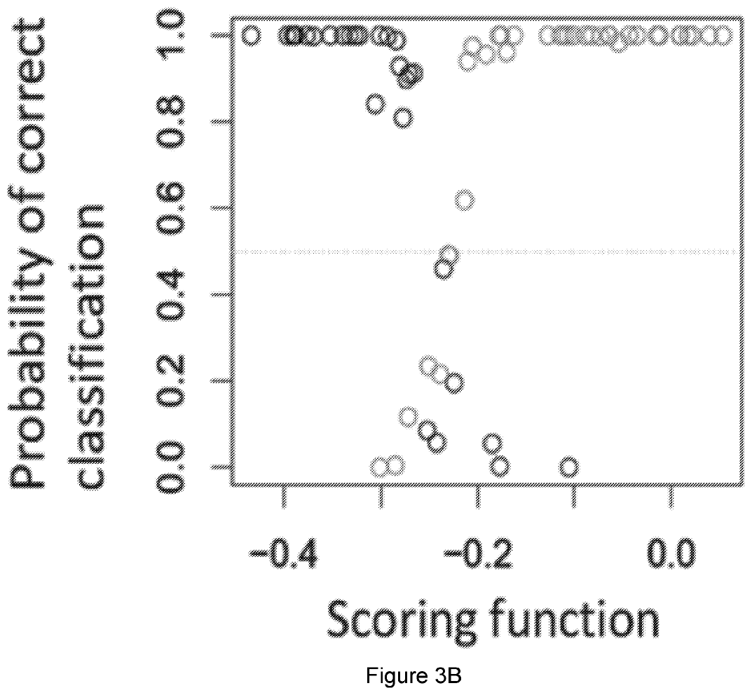

FIGS. 3A and 3B collectively illustrate the classification of patient samples derived from blood plasma with regard to polyp diagnoses, in accordance with some embodiments of the present disclosure.

A classifier was trained against 36 protein profiles of polyp diagnosis vs 28 protein profiles of other benign diagnoses determined from blood plasma using a master list of features, e.g., pairwise comparisons between two protein abundances. For each possible feature set, the dataset was divided into two classes (e.g. Polyps/no Polyps). A classification function that optimizes the separation between given diagnostic classes was then computed as a weighted sum of biomarker levels, where weights are computed analytically using correlations between pairs of selected biomarkers. The training set was used to determine biomarker weights and optimal classification thresholds to be tested in the independent test set.

For each sampling of the test set, a scoring function was computed using sample biomarker's values and weights determined in the training set. Then, classifications was made based on the threshold of the training set. The overall accuracy of classification was assessed in multiple classification tests, where half of a given dataset is used as training set and another half is used as test set. Thus, for each set of a ranked list of candidate features and each sample, the probability of correct classification and average scoring were computed in multiple classification tests. These values were then used for computation of overall classification accuracies assessed by area under receiver operating curve (AUC) both for averaged classification scores and for probabilities.

Expression values of an optimal set of three protein abundance features, HSP90AB1_YARS1, HSP90AB1_MTDH, and HSP90AB1_LYPLA1, were used to train a classifier. The classification accuracies were assessed by area under receiver operating curve (AUC), as illustrated in FIG. 3A. FIG. 3B illustrates averaged classification probabilities as functions of averaged scoring functions. The classification accuracy depends on scoring function and increases at the tails of the distribution. The high degree of consistency between AUCs is derived from scoring function and probability.

### Example 5—Identification of Proteomic Markers for Constructing Classification Signatures to Detect and Classify OvCA Subtypes

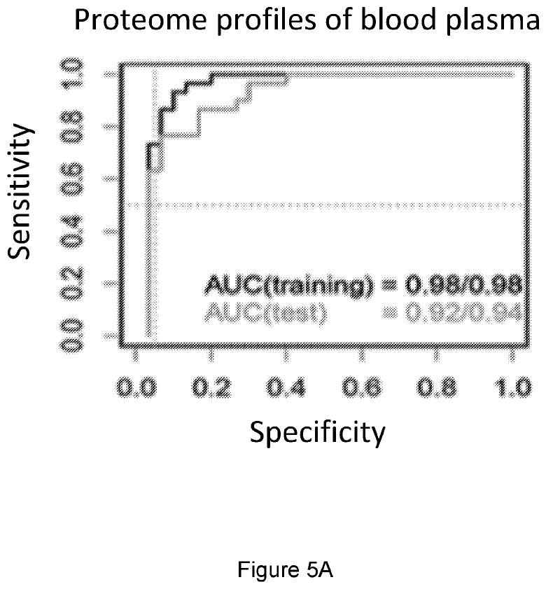

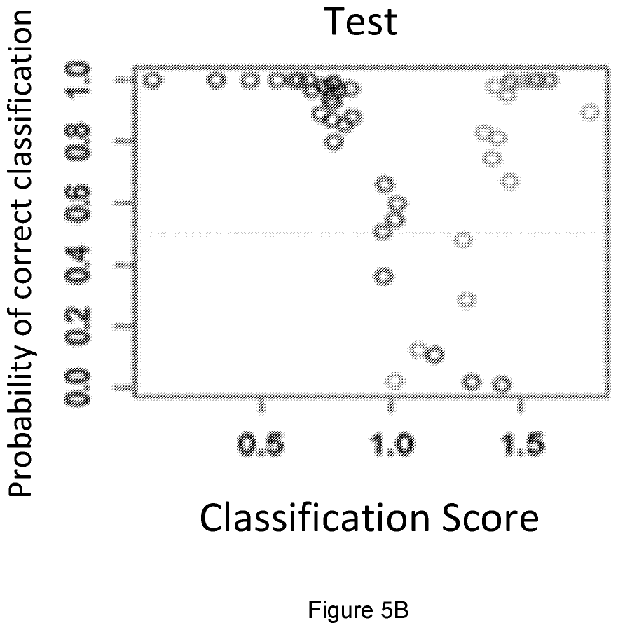

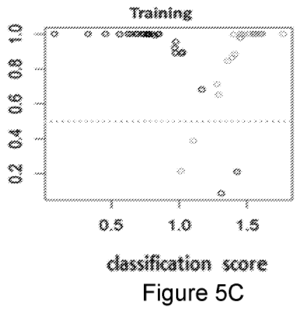

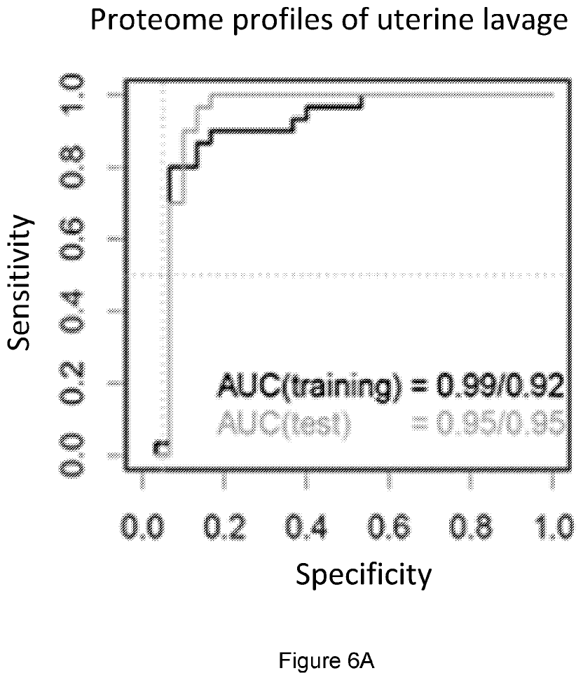

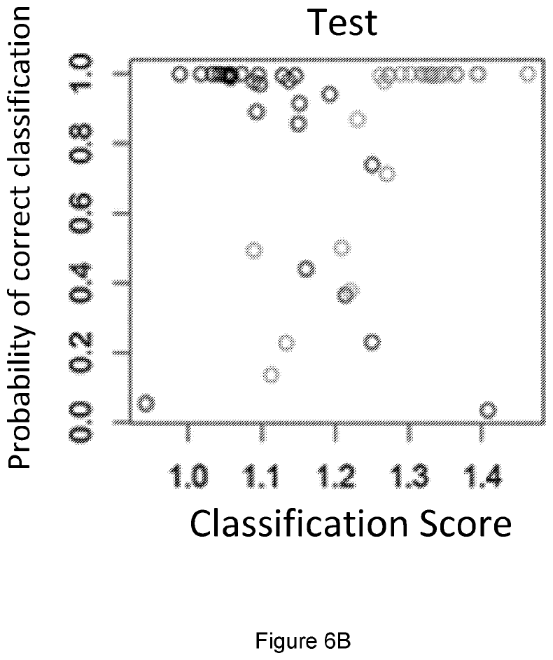

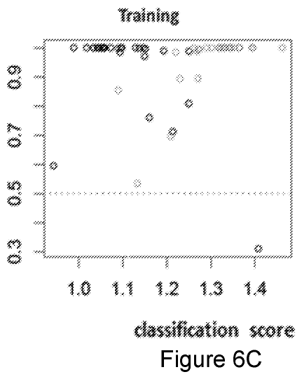

Proteomic data was generated for 120 plasma and lavage samples from women with and without EndoCA. The molecular signature method (MSM) ML-approach described herein was then used to identify a high specificity/sensitivity diagnostic biomarker panel (FIG. 5). Greater than 5,000 proteins were identified in each biofluid. In both lavage and plasma data, classification signatures can be produced on multiple sets of differentially expressed potential biomarkers (>500 proteins can be selected by P<0.01). Fewer than 15 markers were necessary to obtain very high confidence classification accuracies as shown in FIG. 4. Interestingly, the data obtained demonstrated the potential for biological interpretation. In particular, pathway analysis performed on differentially expressed biomarkers of uterine lavage and plasma revealed significant overlapping enrichments of some biomarkers and unique and significant associations specific to each fluid.

To further define robust gynecological classifiers, the MSM algorithm will be used to classify proteome profiles of blood and lavage samples of OvCA patients (150) from those of 200 controls (100 patients with no cancer and 100 patients with EndoCA). Triplicates of ˜30 plasma and lavage profiles will also be used to continue assessing reproducibility. First, the potential of blood and lavage protein profiles to be used for molecular diagnosis of OvCA will be assessed. To do this, classification signatures: OvCA vs benign; OvCA vs EndoCA, OvCA plus EndoCA vs benign, will be derived and examined. This analysis will make it possible to assess and optimize a diagnostic protocol close to real practice cases. Second, the linked clinical annotations of the OvCA samples will be used to determine the potential of protein profiles to classify OvCA by platinum response (sensitive, refractory, resistant). Based on response analysis, a prototype diagnostic panel of optimally selected biomarkers will be developed. Given that DNA and RNAseq data is also linked with the OvCA tumors, future analysis will also allow analysis between tumor molecular data and proteomics.

The MSM approach (FIG. 5) is based on the optimal combination of statistically significant and independent (pairwise correlation <1) biomarkers with relatively low sensitivity. In this context, biomarker refers to a distribution of protein abundance in particular disease subtypes. With this approach, the overall classification accuracy will depend on how well the sensitivities of biomarkers derived from a particular training database reproduce its true population sensitivity. This model estimates that analysis of ˜150 samples for each subtype (OvCA, EndoCA, and benign) will make it possible to reliably determine biomarkers of population sensitivity ˜60% (sensitivity of 50%=random association). In practice, diagnostic power depends on the actual population distribution of biomarkers by sensitivity. This can be illustrated by the following example: a classification function of 5 biomarkers of sensitivity ˜70% can classify only 25% of samples with specificity of 0.95; by adding 10 more biomarkers of sensitivity 60%, ˜50% of samples will be classified with specificity of 0.95; adding 15 more biomarkers of sensitivity 55% will make it possible to classify ˜80% of samples with a specificity of 0.95, and so on. The biomarker sensitivity distributions are not yet well determined, but will be analyzed, practical diagnostics with reliably assessed accuracies will be developed, and larger study sizes will be used to identify all practical biomarkers.

### CONCLUSION

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s) described herein. In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed:

1. A method for evaluating a gynecological disorder in a subject, the method comprising:
a) obtaining a first biological fluid sample from the subject;
b) enriching a protein fraction from the first biological fluid, thereby obtaining a first protein preparation;
c) determining, for each protein in a first set of proteins, a corresponding abundance value for the respective protein in the protein preparation, thereby obtaining a first protein abundance dataset for the subject;
d) determining, using the first protein abundance dataset, values for each of a first set of protein abundance features, thereby obtaining a first feature dataset for the subject; and
e) inputting the first feature set into a classifier trained to distinguish between at least two states of the gynecological disorder based on at least the first set of protein abundance features, thereby obtaining a probability or likelihood from the classifier that the subject has a particular state of a gynecological disorder.

2. The method of claim 1, wherein the first biological fluid sample comprises blood, bone marrow, urine, ascites, sputum, saliva, urine, cerebrospinal fluid, peritoneal fluid, pleural fluid, feces, lymph fluid, gynecological fluids, skin swab, vaginal swab, oral swab, nasal swab, feces, uterine lavage fluid, bladder lavage fluid, oral rinse, or lung washings.

3. The method of claim 1, wherein the first biological fluid sample is a uterine lavage fluid.

4. The method of any one of claims 1-3, wherein the first set of proteins comprises at least 5 proteins selected from the proteins listed in Table 1.

5. The method of any one of claims 1-3, wherein the first set of proteins comprises at least 5 proteins selected from the proteins listed in Table 2.

6. The method of any one of claims 1-3, wherein the first set of proteins comprises at least 5 proteins selected from the proteins listed in Table 3.

7. The method of any one of claims 1-3, wherein the first set of proteins comprises at least 5 proteins selected from the proteins listed in Table 4.

8. The method of any one of claims 1-7, wherein each respective feature in the first set of protein abundance features comprises a normalized abundance value for a respective protein in the first set of proteins.

9. The method of any one of claims 1-7, wherein each respective feature in the first set of protein abundance features comprises a comparison between an abundance value for a first respective protein in the first set of proteins and an abundance value for a second respective protein in the first set of proteins.

10. The method according to any one of claims 1-9, wherein the first set of protein abundance features was determined by a feature selection method comprising (i) defining a list of possible biomarkers, (ii) using a boosting technique to rank the biomarkers, and (iii) performing a plurality of classifications tests to determine a classification signature.

11. The method according to any one of claims 1-10, wherein the classifier determines a disease profile Vₛ for the subject comprising a weighted sum Wₛ of the respective values for each of the first set of protein abundance features in the first feature dataset, calculated as:
Wₛ=Σᵢ₌₁^{m}(AᵢEᵢ),
where:
Eᵢ is a value of a respective protein abundance feature i, in the first feature dataset having m protein abundance features, determined for the first protein abundance dataset, and
Aᵢ is a weight for protein abundance feature i.

12. The method of claim 11, wherein, for each respective protein abundance features i in the first set of m protein abundance features, the weight Aᵢ is calculated as:
Aᵢ˜Dᵢ⁻¹Σⱼ₌₁^{k}([Cᵢⱼ]⁻¹Zⱼ),
where:
Dᵢ is the standard deviation of the value of the protein abundance feature i in a training set of biological fluid samples, wherein the training set comprises:
a first subset of biological fluid samples from training subjects having a first state of the gynecological disorder, and
a second subset of biological fluid samples from training subjects having a second state of the gynecological disorder;
Cᵢⱼ, is a matrix of pairwise correlation between the values of autoantibody abundance features i and j in the first training set, such that [Cᵢⱼ]⁻¹ is the reciprocal matrix of pairwise correlation, wherein k=m−1; and
Zⱼ is a z-score for the values of protein abundance feature j in the first training set, calculated as:
$${Z}_{j} = \frac{{〈 {E}_{j} 〉}_{1} - {〈 {E}_{j} 〉}_{2}}{{D}_{j}} ,$$
where: [symbol]
Eⱼ [symbol] ₁ is the average value of protein abundance feature j determined for the first subset of biological fluid samples, [symbol]
Eⱼ [symbol] ₂ is the average value of protein abundance feature j determined for the second subset of biological fluid samples, and
Dⱼ is the standard deviation of the values of protein abundance feature j determined for the training set of biological fluid samples.

13. The method according to any one of claims 1-12, wherein the classifier comprises a molecular signature algorithm, a neural network algorithm, a support vector machine algorithm, a decision tree algorithm, an unsupervised clustering model algorithm, a supervised clustering model algorithm, or a regression model.

14. The method of any one of claims 1-13, wherein the classifier was trained to distinguish between the at least two states of the gynecological disorder based on at least the values for each of a first set of protein abundance features and one or more secondary features for the subject.

15. The method of claim 14, wherein:
the gynecological disorder condition is an ovarian cancer or an endometrial cancer, and
the one or more secondary features of the subject comprise two or more of the features selected from the group consisting of an age of the subject, a pregnancy history of the subject, a breastfeeding history of the subject, a BRCA1 genotype of the subject, a BRCA2 genotype of the subject, a breast cancer history of the subject, and a familial history of endometrial cancer, ovarian cancer, or breast cancer.

16. The method of any one of claims 1-15, the method further comprising:
obtaining a second biological sample from the subject;
determining a plurality of secondary features from the second biological sample, thereby obtaining a second feature dataset for the subject; and
inputting the second feature dataset into the classifier.

17. The method of claim 16, wherein the second biological sample is a fluid biological sample.

18. The method of claim 16, wherein the second biological sample is a blood plasma sample.

19. The method of any one of claims 1-18, wherein the gynecological disorder is an ovarian cancer or an endometrial cancer.

20. The method of claim 19, wherein the first set of proteins comprises at least 5 proteins selected from the proteins listed in Table 3.

21. The method of claim 19, wherein the first set of proteins comprises at least 5 proteins selected from the proteins listed in Table 4.

22. The method of any one of claims 19-21, wherein the classifier was trained to distinguish between (i) the presence of an ovarian cancer or uterine cancer and (ii) the absence of the ovarian cancer or the uterine cancer, the method further comprising:
when the probability or likelihood obtained from the classifier indicates that the subject has the ovarian cancer or the uterine cancer, administering a therapy for the ovarian cancer or the uterine cancer to the subject, and
when the probability or likelihood obtained from the classifier indicates that the subject does not have the ovarian cancer or the uterine cancer, forgoing administration of the therapy for the ovarian cancer or the uterine cancer to the subject.

23. The method of claim 19, wherein the classifier was trained to distinguish between (i) a first stage of an ovarian cancer or uterine cancer and (ii) a second stage of the ovarian cancer or the uterine cancer that is more advanced than the first stage of the ovarian cancer or the uterine cancer, the method further comprising:
when the probability or likelihood obtained from the classifier indicates that the subject has the first stage of the ovarian cancer or the uterine cancer, administering a first therapy for the ovarian cancer or the uterine cancer to the subject, and
when the probability or likelihood obtained from the classifier indicates that the subject has the first stage of the ovarian cancer or the uterine cancer, administering a second therapy for the ovarian cancer or the uterine cancer to the subject.

24. The method of any one of claims 1-18, wherein the gynecological disorder is adenomyosis, endometrial polyps, leiomyoma, or endometriosis.

25. The method of claim 24, wherein the classifier was trained to distinguish between (i) the presence of adenomyosis, endometrial polyps, leiomyoma, or endometriosis and (ii) the absence of the adenomyosis, endometrial polyps, leiomyoma, or endometriosis, the method further comprising:
when the probability or likelihood obtained from the classifier indicates that the subject has the adenomyosis, endometrial polyps, leiomyoma, or endometriosis, administering a therapy for the adenomyosis, endometrial polyps, leiomyoma, or endometriosis to the subject, and
when the probability or likelihood obtained from the classifier indicates that the subject does not have the adenomyosis, endometrial polyps, leiomyoma, or endometriosis, forgoing administration of the therapy for the adenomyosis, endometrial polyps, leiomyoma, or endometriosis to the subject.

26. The method of any one of claims 1-25, wherein the subject is asymptomatic.

27. The method of any one of claims 1-25, wherein the subject is experiencing pelvic pain, abnormal bleeding, or infertility.
